SEQUENCE LISTING (44 sequences)

SEQ ID NO: 1 - RNA, 25 nt, Artificial Sequence
  G-353
ggcuuuggagacuccguggaggagg

SEQ ID NO: 2 - RNA, 20 nt, Artificial Sequence
  G-644
gguaauaaaggagcuggugg

SEQ ID NO: 3 - RNA, 26 nt, Artificial Sequence
  G-1463
gguggucgcacuauugccuuuggagg

SEQ ID NO: 4 - RNA, 26 nt, Artificial Sequence
  G-1574
gguguuguuggagaagguuccgaagg

SEQ ID NO: 5 - RNA, 29 nt, Artificial Sequence
  G-2714
ggcggugcaccaacaaagguuacuuuugg

SEQ ID NO: 6 - RNA, 17 nt, Artificial Sequence
  G-3467
ggaggagguguugcagg

SEQ ID NO: 7 - RNA, 27 nt, Artificial Sequence
  G-4162
gguuauaccuacuaaaaaggcuggugg

SEQ ID NO: 8 - RNA, 29 nt, Artificial Sequence
  G-4261
ggguuuaaaugguuacacuguagaggagg

SEQ ID NO: 9 - RNA, 23 nt, Artificial Sequence
  G-8687
ggauacaaggcuauugauggugg

SEQ ID NO: 10 - RNA, 30 nt, Artificial Sequence
  G-10267
ggcugguaauguucaacucaggguuauugg

SEQ ID NO: 11 - RNA, 20 nt, Artificial Sequence
  G-13385
gguauguggaaagguuaugg

SEQ ID NO: 12 - RNA, 28 nt, Artificial Sequence
  G-14947
gguuuuccauuuaauaaaugggguaagg

SEQ ID NO: 13 - RNA, 27 nt, Artificial Sequence
  G-15208
ggaacaagcaaauucuauggugguugg

SEQ ID NO: 14 - RNA, 29 nt, Artificial Sequence
  G-15448
ggcgguucacuauauguuaaaccaggugg

SEQ ID NO: 15 - RNA, 23 nt, Artificial Sequence
  G-18296
ggauuggcuucgaugucgagggg

SEQ ID NO: 16 - RNA, 29 nt, Artificial Sequence
  G-22316
ggugauucuucuucagguuggacagcugg

SEQ ID NO: 17 - RNA, 20 nt, Artificial Sequence
  G-24215
gguuggaccuuuggugcagg

SEQ ID NO: 18 - RNA, 24 nt, Artificial Sequence
  G-24268
ggcuuauagguuuaaugguauugg

SEQ ID NO: 19 - RNA, 22 nt, Artificial Sequence
  G-25197
ggccaugguacauuuggcuagg

SEQ ID NO: 20 - RNA, 29 nt, Artificial Sequence
  G-25951
ggugguuauacugaaaaaugggaaucugg

SEQ ID NO: 21 - RNA, 30 nt, Artificial Sequence
  G-26746
ggaucaccgguggaauugcuaucgcaaugg

SEQ ID NO: 22 - RNA, 29 nt, Artificial Sequence
  G-28781
ggcuucuacgcagaagggagcagaggcgg

SEQ ID NO: 23 - RNA, 15 nt, Artificial Sequence
  G-28903
ggcuggcaauggcgg

SEQ ID NO: 24 - RNA, 19 nt, Artificial Sequence
  G-29123
ggaaauuuuggggaccagg

SEQ ID NO: 25 - RNA, 30 nt, Artificial Sequence; identical to SEQ ID NO: 10
  G-29234

SEQ ID NO: 26 - DNA, 27 bp, Artificial Sequence
  WT G4-353 (Fwd)
tatagctcgagatggagagccttgtcc

SEQ ID NO: 27 - DNA, 28 bp, Artificial Sequence
  WT G4-353 (Rev)
tatacggatccacatgaccatgaggtgc

SEQ ID NO: 28 - DNA, 28 bp, Artificial Sequence
  WT G4-644 (Fwd)
tatacctcgagatggttgagctggtagc

SEQ ID NO: 29 - DNA, 26 bp, Artificial Sequence
  WT G4-644 (Rev)
tatacggatccccgttaagctcacgc

SEQ ID NO: 30 - DNA, 37 bp, Artificial Sequence
  WT G4-3467 (Fwd)
gataactcgagatggaacttacaccagttgttcagac

SEQ ID NO: 31 - DNA, 35 bp, Artificial Sequence
  WT G4-3467 (Rev)
aatataggatccggaatctcagcgatcttttgttc

SEQ ID NO: 32 - DNA, 45 bp, Artificial Sequence
  Mut G4-353 (Fwd)
cgcgacgtgctcgtacgtgactttgaagactccgtagaagaagtc

SEQ ID NO: 33 - DNA, 45 bp, Artificial Sequence
  Mut G4-353 (Rev)
gacttcttctacggagtcttcaaagtcacgtacgagcacgtcgcg

SEQ ID NO: 34 - DNA, 38 bp, Artificial Sequence
  Mut G4-644 (Fwd)
cggtaataaaagagctagtagccatagttacggcgccg

SEQ ID NO: 35 - DNA, 38 bp, Artificial Sequence
  Mut G4-644 (Rev)
cggcgccgtaactatggctactagctcttttattaccg

SEQ ID NO: 36 - DNA, 38 bp, Artificial Sequence
  Mut G4-3467 (Fwd)
catggaagaagtgttgcaagagccttaaataaggctac

SEQ ID NO: 37 - DNA, 38 bp, Artificial Sequence
  Mut G4-3467 (Rev)
gtagccttatttaaggctcttgcaacacttcttccatg

SEQ ID NO: 38 - DNA, 38 bp, Artificial Sequence
  T7-G4-353 (Fwd)
agtaatacgactcactatagggatggagagccttgtcc

SEQ ID NO: 39 - DNA, 41 bp, Artificial Sequence
  T7-G4-644 (Fwd)
agtaatacgactcactatagggatggttgagctggtagcag

SEQ ID NO: 40 - DNA, 44 bp, Artificial Sequence
  T7-G4-3467 (Fwd)
agtaatacgactcactatagggatggaacttacaccagttgttc

SEQ ID NO: 41 - DNA, 22 bp, Artificial Sequence
  T7-EYFP (Rev)
ttacttgtacagctcgtccatg

SEQ ID NO: 42 - RNA, 25 nt, Artificial Sequence
  Mut-G-353
gacuuugaagacuccguagaagaag

SEQ ID NO: 43 - RNA, 20 nt, Artificial Sequence
  Mut-G-644
gguaauaaaagagcuaguag

SEQ ID NO: 44 - RNA, 17 nt, Artificial Sequence
  Mut-G-3467
ggaagaaguguugcaag

(19) United States
(12) Patent Application Publication
(10) Pub. No.: US 2024/0390328 A1
(43) Pub. Date: Nov. 28, 2024

(54) PHARMACEUTICAL COMPOSITION CONTAINING AS ACTIVE INGREDIENT COMPOUND CONJUGATED TO G4 STRUCTURES OF CORONAVIRUSES FOR PREVENTION OR TREATMENT OF CORONAVIRUS INFECTIOUS DISEASES

(71) Applicants: RESEARCH & BUSINESS FOUNDATION SUNGKYUNKWAN UNIVERSITY, Suwon-si (KR); KOREA UNIVERSITY RESEARCH AND BUSINESS FOUNDATION, Seoul (KR)

(72) Inventors: Kyeong Kyu KIM, Seoul (KR); Moon Jung SONG, Seoul (KR); Man-Seong PARK, Seoul (KR); Jin-Hyun AHN, Suwon-si (KR); Maria RAZZAQ, Suwon-si (KR); Subramaniyam RAVICHANDRAN, Suwon-si (KR); Ji Ho HAN, Seoul (KR); Jae Hyun KIM, Uijeongbu-si (KR)

(73) Assignees: RESEARCH & BUSINESS FOUNDATION SUNGKYUNKWAN UNIVERSITY, Suwon-si (KR); KOREA UNIVERSITY RESEARCH AND BUSINESS FOUNDATION, Seoul (KR)

(21) Appl. No.: 18/245,941

(22) PCT Filed: Sep. 17, 2021
(86) PCT No.: PCT/KR2021/095084

§ 371 Date: Mar. 20, 2023

(30) Foreign Application Priority Data
Sep. 18, 2020 (KR) 10-2020-0120799
Aug. 6, 2021 (KR) 10-2021-0104190

(51) Int. Cl. A61K 31/409; A61K 31/444; A61K 31/4709; A61K 31/473; A61K 31/4745; A61K 31/5383; A61P 31/14

(52) U.S. Cl. CPC A61K 31/409; A61K 31/444; A61K 31/4709; A61K 31/473; A61K 31/4745; A61K 31/5383; A61P 31/14

(57) ABSTRACT

The present invention relates to a composition for preventing or treating coronavirus infectious diseases, comprising a compound that binds to G4 structures of coronaviruses. Since the compound according to the present invention can bind to G4 structures of coronaviruses to stabilize the G4 structures, reduce the protein expression of coronaviruses, and effectively inhibit cell infection by viruses, the compound is expected to be used in the prevention or treatment of coronavirus infectious diseases.

### TECHNICAL FIELD

The present invention relates to a composition for preventing or treating coronavirus infectious diseases, comprising a compound that binds to G4 structures of coronaviruses.

The present application claims priority to and the benefit of Korean Patent Application Nos. 10-2020-0120799 and 10-2020-0104190 filed in the Korean Intellectual Property Office on Sep. 18, 2020 and Aug. 6, 2021, respectively, and all the contents disclosed in the specification and drawings of those applications are incorporated in the present application.

### BACKGROUND ART

Severe acute respiratory syndrome coronavirus 2 (SARS-COV-2 or 2019-nCOV), which caused a worldwide coronavirus infectious disease (COVID-19) pandemic centered in Wuhan, China, was reported to have caused 190,877,071 confirmed cases and 4,095,650 deaths as of Jul. 20, 2021. Accordingly, there is a worldwide need for therapeutic and preventive methods.

SARS-COV-2 (2019-nCOV) is known to enter cells by utilizing angiotensin-converting enzyme 2 (ACE2) on the surface of human body cells as a receptor. ACE2 is widely distributed mainly in the lungs, arteries, kidneys, heart, small intestine, and the like, and is known to be transmitted through the respiratory system and induce severe acute respiratory syndrome (SARS). In the case of SARS-COV-2 (2019-nCOV), various subunits of a spike-shaped protein called the spike(S) protein are understood to interact with the ACE2 of host cells to infect the host. Most therapeutic agents currently under development are made using small molecules that bind to viral spike proteins, protein hydrolases or nucleic acid polymerases.

Numerous SARS-COV-2 variants have emerged and circulated globally throughout the COVID-19 pandemic. Most of these variants exhibit SARS-COV-2 spike protein mutations such as the N501Y, E484K and D614G mutations. The spike protein is a target of most COVID-19 vaccines, but many mutants exhibit a high transmission rate and have the ability to evade neutralizing antibodies. Although prophylactic vaccines and drugs that target viral proteins are fundamental methods of combating viral infections, targeting a conserved genomic site within the same virus family can offer a new opportunity to develop a new antiviral therapy.

Intracellular DNA generally has a double helix structure, but is known to have various structures in a specific cellular environment or during a specific cellular action. Among such special structures, a quadruple helix structure formed by four-stranded DNA or RNA is well known, and such a structure is called a G-quadruplex (G4) or G4 structure. The G4 structure is well formed when a sequence of guanines are present in DNA or RNA bases. The guanines of a DNA strand bind to form a square plate structure, and such plates are stacked one after another to form a stable knot in the shape of a hexahedron. The G4 structure is known to regulate gene expression, DNA replication or protein translation processes.

Against this background, the present inventors have studied 25 G4 structures present in coronaviruses, and confirmed through the stabilization of the G4 structure by binding to a compound that the expression of major proteins of coronaviruses can be effectively inhibited and viral cell infection can be effectively inhibited, thereby completing the present invention.

### DISCLOSURE

### Technical Problem

The present inventors confirmed the effect of preventing or treating coronavirus infectious diseases by confirming through the stabilization of the G4 structures by binding to a compound that the expression of major proteins of coronaviruses can be effectively inhibited and viral cell infection can be effectively inhibited while studying 25 G4 structures present in coronaviruses, thereby completing the present invention based on this.

Thus, an object of the present invention is to provide a pharmaceutical composition for preventing or treating coronavirus infectious diseases, comprising, as an active ingredient, a compound that binds to G4 structures of coronaviruses or a pharmaceutically acceptable salt thereof.

Another object of the present invention is to provide a food composition for preventing or ameliorating coronavirus infectious diseases, comprising, as an active ingredient, a compound that binds to G4 structures of coronaviruses or a pharmaceutically acceptable salt thereof.

Still another object of the present invention is to provide a quasi-drug composition for preventing or suppressing coronavirus infectious diseases, comprising a compound that binds to G4 structures of coronaviruses as an active ingredient.

Yet another object of the present invention is to provide an antiviral composition against coronaviruses, comprising a compound that binds to G4 structures of coronaviruses as an active ingredient.

However, the technical problems which the present invention intends to solve are not limited to the technical problems that have been mentioned above, and other technical problems which have not been mentioned will be clearly understood by a person with ordinary skill in the art to which the present invention pertains from the following description.

### Technical Solution

To achieve the objects of the present invention, the present invention provides a pharmaceutical composition for preventing or treating coronavirus infectious diseases, comprising, as an active ingredient, a compound that binds to G4 structures of coronaviruses or a pharmaceutically acceptable salt thereof.

Further, the present invention provides a food composition for preventing or ameliorating coronavirus infectious diseases, comprising, as an active ingredient, a compound that binds to G4 structures of coronaviruses or a sitologically acceptable salt thereof.

In addition, the present invention provides a quasi-drug composition for preventing or suppressing coronavirus infectious diseases, comprising a compound that binds to G4 structures of coronaviruses as an active ingredient.

Furthermore, the present invention provides an antiviral composition against coronaviruses, comprising a compound that binds to G4 structures of coronaviruses as an active ingredient.

Further, the present invention provides a method for preventing or treating coronavirus infectious diseases, the method comprising administering a pharmaceutical composition comprising, as an active ingredient, a compound that binds to G4 structures of coronaviruses or a pharmaceutically acceptable salt thereof to a subject in need thereof.

In addition, the present invention provides a use of a pharmaceutical composition comprising, as an active ingredient, a compound that binds to G4 structures of coronaviruses or a pharmaceutically acceptable salt thereof for preventing, ameliorating or treating coronavirus infectious diseases.

Furthermore, the present invention provides a use of a compound that binds to G4 structures of coronaviruses or a pharmaceutically acceptable salt thereof for producing a preventive or therapeutic agent for coronavirus infectious diseases.

Further, the present invention provides a method for suppressing coronaviruses, the method comprising applying a pharmaceutical composition comprising, as an active ingredient, a compound that binds to G4 structures of coronaviruses or a pharmaceutically acceptable salt thereof to a subject.

In addition, the present invention provides an antiviral use of a composition comprising, as an active ingredient, a compound that binds to G4 structures of coronaviruses or a pharmaceutically acceptable salt thereof against coronaviruses.

Furthermore, the present invention provides a use of a compound that binds to G4 structures of coronaviruses or a pharmaceutically acceptable salt thereof for producing an antiviral preparation against coronaviruses.

In an exemplary embodiment of the present invention, the compound may be one or more selected from the group consisting of compounds represented by the following Chemical Formulae 1 to 7, but is not limited thereto.

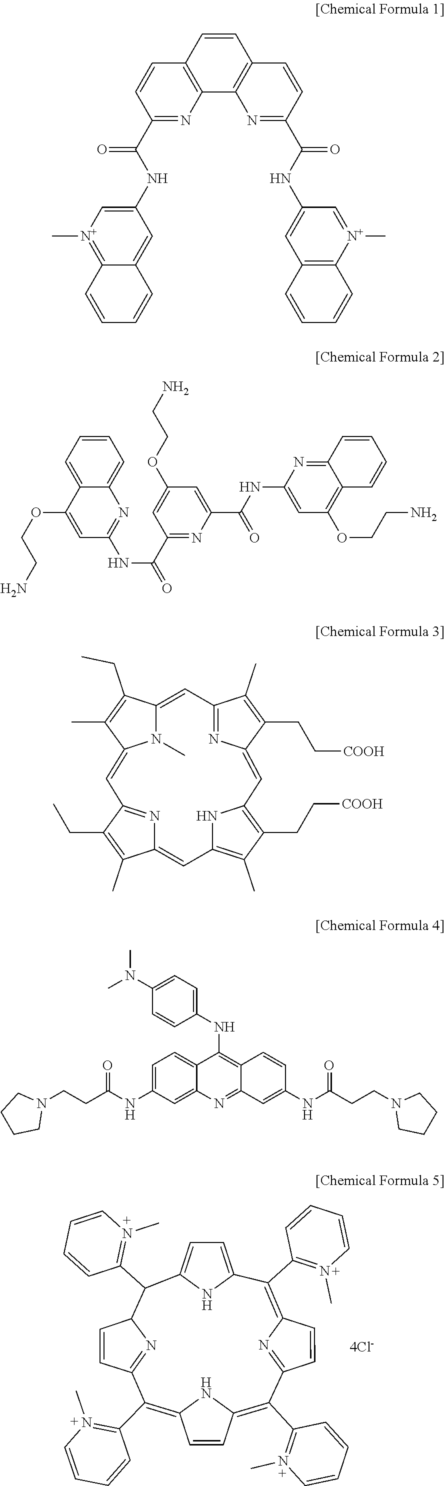

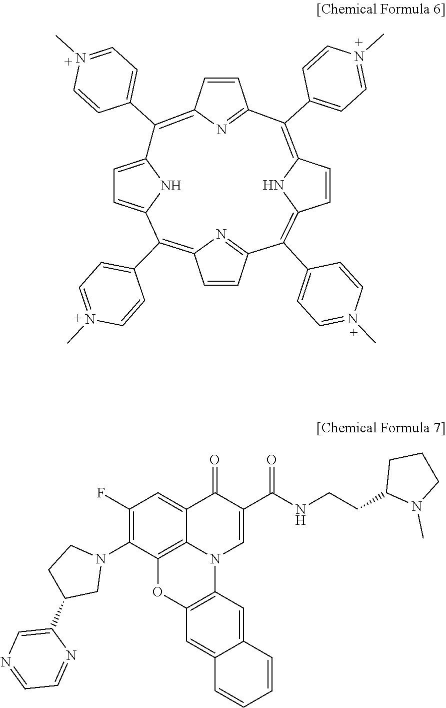

In another exemplary embodiment of the present invention, the coronavirus may be one or more selected from the group consisting of human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, Middle East respiratory syndrome coronavirus (MERS-COV), and severe acute respiratory syndrome coronavirus 2 (SARS-COV-2), but is not limited thereto.

In still another exemplary embodiment of the present invention, the compound may be characterized by stabilizing G4 structures of coronaviruses, but is not limited thereto.

In yet another exemplary embodiment of the present invention, the compound may be characterized by suppressing the expression of one or more proteins selected from the group consisting of coronavirus protein non-structural protein 1 (Nsp1), non-structural protein 3 (Nsp3), and a nucleocapsid protein, but is not limited thereto.

In yet another exemplary embodiment of the present invention, the compound may be characterized by suppressing coronavirus virion production, but is not limited thereto.

In yet another exemplary embodiment of the present invention, the quasi-drug may be characterized by being one or more selected from the group consisting of an antiseptic cleaner, shower foam, a mouthwash, a wet wipe, a detergent soap, a hand wash, and an ointment, but is not limited thereto.

### Advantageous Effects

Since the compound according to the present invention can bind to G4 structures of coronaviruses to stabilize the G4 structure, reduce the protein expression of coronavirus, and effectively inhibit cell infection by viruses, the compound can be used in the prevention or treatment of coronavirus infectious diseases.

### DESCRIPTION OF DRAWINGS

FIG. 1 illustrates the distribution of genes and G4 of SARS-COV-2.

FIGS. 2A and 2B illustrate the CD spectra of 25 G4s present in coronaviruses. The x-axis indicates wavelength (nm) and the y-axis indicates ellipticity (mdeg).

FIGS. 3A and 3B illustrate the thermal melting curves of 25 G4s present in coronaviruses. The x-axis indicates wavelength (nm) and the y-axis indicates normalized ellipticity (mdeg).

FIG. 4 illustrates CD spectra and thermal melting curves of WT-G-353, WT-G-644 and WT-G-3467 and Mut-G-353, Mut-G-644 and Mut-G-3467.

FIGS. 5A and 5B illustrate the CD spectra of 25 G4s present in coronaviruses with and without the binding of CX-3543, PDS and PhenDC3, which are G4-binding compounds.

FIGS. 6A and 6B illustrate the thermal melting curves of 25 G4s present in coronaviruses with and without the binding of CX-3543, PDS and PhenDC3, which are G4-binding compounds.

FIG. 7A illustrates the cell viability when HEK293T cells were treated with 0.1, 0.5, 1, 2.5, 5.5, 7.5, 10, 25, 50 and 75 μM PDS. The x-axis indicates concentration (μM) and the y-axis indicates viability.

FIG. 7B illustrates the cell viability when Vero cells were treated with 0.1, 0.5, 1, 2.5, 5.5, 7.5, 10, 25, 50 and 75 μM PDS. The x-axis indicates concentration (μM) and the y-axis indicates viability.

FIG. 7C illustrates the cell viability when HEK293T cells were treated with 0.1, 0.5, 1, 2.5, 5.5, 7.5, 10, 25, 50 and 75 M PhenDC3. The x-axis indicates concentration (μM) and the y-axis indicates viability.

FIG. 7D illustrates the cell viability when Vero cells were treated with 0.1, 0.5, 1, 2.5, 5.5, 7.5, 10, 25, 50 and 75 μM PhenDC3. The x-axis indicates concentration (μM) and the y-axis indicates viability.

FIG. 8 illustrates the results of gradient PCR performed on T7-WT-G-353, T7-WT-G-644, T7-WT-G-3467, T7-Mut-G-353, T7-Mut-G-644 and T7-Mut-G-3467 at 61, 63, 65, 67, and 69° C. M indicates a 1 kb DNA marker in a range of 250 bp to 10 kb.

FIGS. 9A to 9C illustrate IVT analysis results as band intensity graphs of a western blot when N1-WT-G-353, N1-WT-G-644 and N1-WT-G-3467 were treated with 5, 15, 25 and 50 μM PDS, which is a G4-binding compound. The x-axis indicates concentration (μM) and the y-axis indicates band intensity.

FIGS. 10A to 10C illustrate IVT analysis results as band intensity graphs of a western blot when N1-WT-G-353, N1-WT-G-644 and N1-WT-G-3467 were treated with 5, 15, 25 and 50 μM PhenDC3, which is a G4-binding compound. The x-axis indicates concentration (μM) and the y-axis indicates band intensity.

FIGS. 11A and 11B illustrate IVT analysis results as a western blot when N1-WT-G-353, N1-WT-G-644 and N1-WT-G-3467 were treated with 5, 10, 15, 20, 25 and 50 μM PDS, which is a G4-binding compound.

FIG. 11C illustrates IVT analysis results as a western blot when N1-WT-G-3467 was treated with 5, 15, 25 and 50 μM PDS, which is a G4-binding compound.

FIG. 11D illustrates IVT analysis results as a western blot when N1-Mut-G-3467 was treated with 5, 15, 25 and 50 μM PDS, which is a G4-binding compound.

FIGS. 12A and 12B illustrate IVT analysis results as a western blot when N1-WT-G-353 and N1-WT-G-644 were treated with 5, 10, 15, 20, 25 and 50 μM PhenDC3, which is a G4-binding compound.

FIG. 12C illustrates IVT analysis results as a western blot when N1-WT-G-3467 was treated with 5, 15, 25 and 50 μM PhenDC3, which is a G4-binding compound.

FIG. 12D illustrates IVT analysis results as a western blot when N1-Mut-G-3467 was treated with 5, 15, 25 and 50 μM PhenDC3, which is a G4-binding compound.

FIG. 13 illustrates the antiviral activity against N protein expression in Vero cells infected with SARS-COV-2 as a dose-response curve analysis result of inhibition of SARS-COV-2 infection according to the concentration of NMM, PDS, PhenDC3, and BRACO19, which are G4-binding compounds. ● indicates the SARS-COV-2 infection inhibition rate of the corresponding compound. ▪ indicates the cytotoxicity of each compound, and the cell number in each well was normalized to the average cell number in the mock group wells, and denoted as ‘cell number to mock’ on the graph.

FIG. 14A illustrates the antiviral activity against virion production in a culture solution of Vero cells infected with SARS-COV-2 as a dose-response curve analysis result of SARS-COV-2 infection inhibition according to the concentration of PDS, which is a G4-binding compound. The x-axis indicates concentration (log μM) and the y-axis indicates the infection inhibition rate (%) of SARS-COV-2.

FIG. 14B illustrates the antiviral activity against virion production in a culture solution of Vero cells infected with SARS-COV-2 as a dose-response curve analysis result of SARS-COV-2 infection inhibition according to the concentration of PhenDC3, which is a G4-binding compound. The x-axis indicates concentration (log μM) and the y-axis indicates the infection inhibition rate (%) of SARS-COV-2.

FIG. 15 illustrates the antiviral activity against virion production in a culture solution of Vero cells infected with SARS-COV-2 as a dose-response curve analysis result of SARS-COV-2 infection inhibition according to the concentration of NMM, which is a G4-binding compound. The x-axis indicates concentration (log μM) and the y-axis indicates the infection inhibition rate (%) of SARS-COV-2.

FIG. 16 illustrates the antiviral activity against virion production in HCT-8 cells infected with the HCoV-OC43 virus as a dose-response curve analysis result of HCoV-OC43 virus infection inhibition according to the concentration of NMM, TMPyP2, TMPyP4, or CX3543, which is a G4-binding compound. The x-axis indicates concentration (log μM), and the y-axis indicates the infection inhibition rate (%) and cell viability (%) of the HCoV-OC43 virus. The table in FIG. 16 shows the IC₅₀ values for each G4-binding compound. A dark red ● indicates the cytotoxicity of each compound in HCT-8 cells, and the cytotoxicity of each compound at each concentration was expressed as relative cell viability (%) in the graph by normalizing a group treated with the G4-binding compound to the DMSO or distilled water control.

### MODES OF THE INVENTION

The inventors of the present invention confirmed that the compound can bind to G4 structures of coronaviruses to stabilize the G4 structures and reduce the protein expression of coronaviruses, thereby completing the present invention.

In an exemplary embodiment of the present invention, compounds PhenDC3 and PDS were confirmed to stabilize G4 through CD spectra and thermal melting curves according to the presence or absence of the G4-binding compound (see Example 4).

In another exemplary embodiment of the present invention, it was confirmed that compounds PhenDC3 and PDS were not cytotoxic in HEK293T and Vero cells (see Example 5).

In still another exemplary embodiment of the present invention, it was confirmed through in-vitro transcription/translation (IVT) analysis that PDS and PhenDC3, which are G4-binding compounds, suppress the expression of Nsp1 and Nsp3 (see Example 7).

In yet another exemplary embodiment of the present invention, antiviral activity against N protein expression according to G4-binding compounds (NMM, PDS, PhenDC3, and BRACO19) in cells infected with SARS-COV-2 was confirmed (see Example 8).

In yet another exemplary embodiment of the present invention, antiviral activity against virion production according to G4-binding compounds (PDS, PhenDC3, and NMM) in cells infected with SARS-COV-2 was confirmed (see Example 9).

In yet another exemplary embodiment of the present invention, antiviral activity against virion production according to G4-binding compounds (NMM, TMPyP2, TMPyP4, and CX3543) in cells infected with the HCoV-OC43 virus was confirmed (see Example 10).

Through the results of the examples as described above, it was confirmed that the compound according to the present invention stabilizes G4 structures of coronaviruses and reduces the protein expression of coronaviruses, so that the compound may be used for the prevention or treatment of coronavirus infectious diseases.

Hereinafter, the present invention will be described in detail.

The present invention provides a pharmaceutical composition for preventing or suppressing coronavirus infectious diseases, comprising a compound that binds to G4 structures of coronaviruses as an active ingredient.

In the present invention, the compound may be characterized by being one or more selected from the group consisting of compounds represented by the following Chemical Formulae 1 to 7, but is not limited thereto.

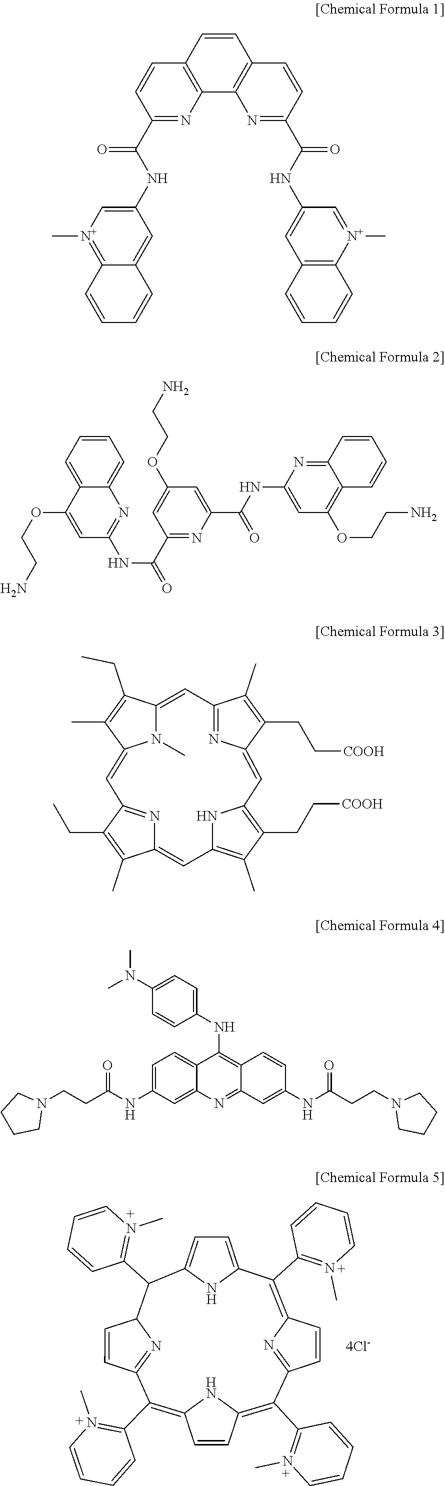

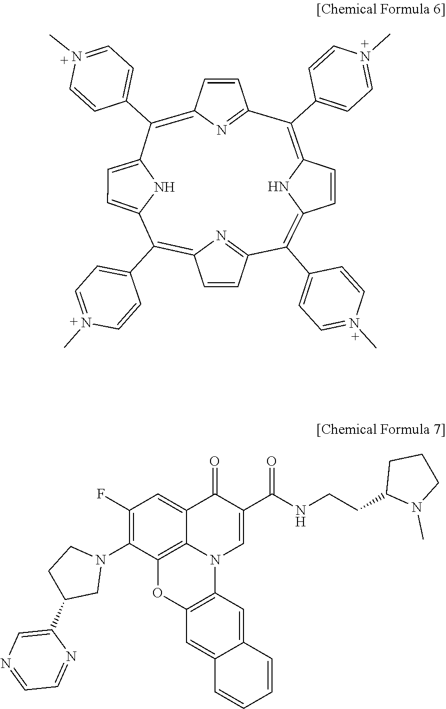

In the present invention, the compound of Chemical Formula 1 may be named PhenDC3, and may have an IUPAC name of 2-N, 9-N-bis(1-methylquinolin-1-ium-3-Further, the compound of Chemical 10 yl)-1,10-phenanthroline-2,9-dicarboxamide. Formula 1 may have a molecular weight of 550.6 and a chemical formula of C₃₄H₂₆N₆O₂.

In the present invention, the compound of Chemical Formula 2 may be named pyridostatin (PDS), and may have an IUPAC name of 4-(2-aminoethoxy)-N2,N6-bis(4-(2-aminoethoxy) quinolin-2-yl)pyridine-2,6-dicarboxamide. In addition, the compound of Chemical Formula 2 may have a molecular weight of 596.6, a chemical formula of C₃₁H₃₂N₈O₅, a boiling point of 753.8±60.0° C., and a flash point of 409.7±32.9° C.

In the present invention, the compound of Chemical Formula 3 may be named N-methyl mesoporphyrin IX (NMM9), and may have an IUPAC name of 3-[18-(2-carboxyethyl)-7,12-diethyl-3,8,13,17,22-pentamethyl-23H-porphyrin-2-yl]propanoic acid. Furthermore, the compound of Chemical Formula 3 may have a molecular weight of 580.7, a chemical formula of C₃₅H₄₀N₄O₄ and a density of 1.26 g/cm³.

In the present invention, the compound of Chemical Formula 4 may be named BRACO-19, and may have an IUPAC name of N-[9-[4-(dimethylamino) anilino]-6-(3-pyrrolidin-1-ylpropanoylamino) acridin-3-yl]-3-pyrrolidin-1-ylpropanamide. Further, the compound of Chemical Formula 4 may have a molecular weight of 593.8, a chemical formula of C₃₅H₄₃N₇O₂, a boiling point of 854.95° C., a flash point of 470.857° C., and a density of 1.275 g/cm³.

In the present invention, the compound of Chemical Formula 5 may be named TMPyP2, and may have an IUPAC name of 5,10,15,20-tetrakis(1-methylpyridin-1-ium-2-yl)-21,23-dihydroporphyrin. In addition, the compound of Chemical Formula 5 may have a molecular weight of 678.8 and a chemical formula of C₄₄H₃₈N₈⁴⁺.

In the present invention, the compound of Chemical Formula 6 may be named TMPyP4, and may have an IUPAC name of 5,10,15,20-tetrakis(1-methylpyridin-1-ium-4-yl)-21,23-dihydroporphyrin. Furthermore, the compound of Chemical Formula 6 may have a molecular weight of 678.8 and a chemical formula of C₄₄H₃₈N₈⁴⁺.

In the present invention, the compound of Chemical Formula 7 may be named CX 3543, and may have an IUPAC name of 15-fluoro-N-[2-[(2S)-1-methylpyrrolidin-2-yl]ethyl]-18-oxo-14-(3-pyrazin-2-ylpyrrolidin-1-yl)-12-oxa-1-azapentacyclo [11.7.1.0^{2,11}.0^{4,9}.0^{17,21}]henicosa-2,4,6,8, 10, 13 (21), 14,16,19-nonaene-19-carboxamide. Further, the compound of Chemical Formula 7 may have a molecular weight of 604.673, a chemical formula of C₃₅H₃₃FN₆O₃, and a boiling point of 845.3±65.0° C.

In the present invention, the coronavirus may be severe acute respiratory syndrome coronavirus 2 (SARS-Cov-2 or 2019 novel coronavirus or 2019-nCOV), but is not limited thereto.

As used herein, the term “coronavirus” refers to a species belonging to the genus Nidovirales, Coronaviridae, Coronavirinae or Torovirinae. Coronaviruses are enveloped viruses with +ssRNA and with a helical-symmetric nucleocapsid. In addition, severe acute respiratory syndrome coronavirus 2 (SARS-COV-2) is also the seventh coronavirus to infect humans so far, and the others are human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, and Middle East respiratory syndrome coronavirus (MERS-COV). The proteins that contribute to the overall structure of coronaviruses are spike, envelope and nucleocapsid proteins. In the case of the SARS coronavirus, the definite binding receptor domain on the spike(S) mediates the attachment portion of the virus to its cell receptor, angiotensin converting enzyme 2 (ACE2). Some coronaviruses (particularly, members of the beta coronavirus subgroup) also have short spikes like proteins called antibody esterases. Coronaviruses may cause viral pneumonia or secondary bacterial pneumonia, and may also cause direct viral bronchitis or secondary bacterial bronchitis. The human coronavirus that was discovered in 2003 is the severe acute respiratory syndrome coronavirus (SARS-COV) that causes severe acute respiratory syndrome (SARS), and causes upper and lower respiratory tract infections.

Furthermore, the term “SARS-COV-2” or “2019-nCOV” as used herein refers to a novel coronavirus, and represents variants of SARS and MERS as RNA viruses. SARS-COV-2 shares a sequence identity of about 79.7% with SARS and a sequence identity of about 50% with MERS. However, in contrast to SARS and MERS, the spike glycoprotein of 2019-nCOV forms a morphological structure in which one RBD domain protrudes upward, and as a result, it exhibits a 100- to 1000-fold stronger binding force with angiotensin (ACE2) which is a target receptor. Such strong binding force facilitates entry into cells, and thus, acts as a cause of increased infectivity.

In the present invention, the coronavirus infectious disease may be a coronavirus respiratory infectious disease. The viral respiratory infectious disease may exhibit symptoms such as coughing, sneezing, a headache, a stuffy nose, a sore throat, diarrhea, discoloration of fingers or toes, conjunctivitis, a high fever, wheezing, bronchitis, bronchiolitis, pneumonia, asthma, loss of smell and taste, and respiratory failure. When the coronavirus is severe acute respiratory syndrome coronavirus 2 (SARS-Cov-2), the main symptoms are a fever and respiratory symptoms (coughing, sore throat, and dyspnea), and it is possible to exhibit symptoms such as a headache, muscle pain, hemoptysis and nausea, chills, chest pain, and diarrhea along with the main symptoms. Further, the coronavirus infectious disease may be coronavirus disease-19 (COVID-19).

In the present invention, the virus may modulate its genome through replication and infection for survival. Viral RNA genomes possess various regions such as structural domains, internal ribosome entry sites (IRES), polycistronic mRNAs, overlapping reading frames and RNA pseudoknot structures to control genome expression and replication. For example, the folding of specific regions of genomic RNA into stable noncanonical structures may act as an obstacle to viral RNA metabolism.

As used herein, the term “spike protein” or “S protein” refers to a coronavirus surface protein.

As used herein, the term “G4 structure” or “G-quadruplex (G4)” refers to a four-stranded nucleic acid secondary structure formed by guanine-rich DNA or RNA sequences. The G4 structure is widely distributed in both DNA and RNA viral genomes. For example, the G4 structure has been discovered and reported in Epstein-Barr virus (EBV), Zika virus (ZIKV), Hepatitis C virus (HCV), human papillomavirus (HPV), human herpes simplex virus (HSV), Ebola virus, human immunodeficiency virus 1 (HIV-1), influenza virus (H1N1) and human cytomegalovirus (HCMV) and long terminal repeat regions of retroviruses and lentiviruses. Recently, bioinformatics analysis of whole genomes and transcripts obtained using G4-specific antibodies and compounds have revealed the genetic location of G4. DNA-G4 plays an important role in telomeres, mitotic and meiotic double-strand break sites, transcription initiation sites and replication origins. RNA-G4 coordinates many steps of RNA metabolism ranging from splicing, RNA processing and transport to mRNA translation (Int. J. Mol. Sci. 2019, 20, 2884).

In the present invention, the compound may be a material that stabilizes G4, but is not limited thereto. For example, the Braco-19 is a compound that stop virus entry and replication by exhibiting inhibitory activity on the expression of the G4 harboring gene in the Nipah virus. In addition, the CX-3543 is the only G4-binding compound that has passed in vivo clinical phase 2 trials for carcinoid and neuroendocrine tumors. Furthermore, the PDS is a compound known to reduce gene expression by stabilizing G4 conserved in EBV, pseudorabies virus and herpes virus miRNAs. Further, the PhenDC3 is a compound known to suppress HCV and EBV virus replication in cells without cytotoxicity.

In the present invention, the compound may be characterized by suppressing the expression of one or more proteins selected from the group consisting of coronavirus protein non-structural protein 1 (Nsp1), non-structural protein 3 (Nsp3), and a nucleocapsid protein, but is not limited thereto.

As used herein, the term “non-structural protein 1 (Nsp1)” is the most important virulence factor that plays multiple roles in interfering with virus replication and host immune responses. In SARS-COV-2, Nsp1 comprises G4-353 and G4-644 in a 540 bp-long RNA open reading frame (ORF). Nsp1 primarily targets type I interferon expression and antiviral signaling pathways to suppress host mRNA translation and suppress host innate immune function.

As used herein, the term “non-structural protein 3 (Nsp3)” refers to the largest multidomain gene (5.83 Kbp) in SARS-COV-2 with multiple functions. The Nsp3-MACI domain is 618 bp long and comprises G4-3467. The Nsp3-MACI domain counteracts host innate immunity through antiviral de-ADP ribosylating activity catalyzed by poly(ADP-ribose) polymerases (PARPs). De-mono-ADP-ribosylation of STAT1 by the Nsp3-Mac1 domain may trigger the inflammatory cytokine storm observed in severe cases of COVID-19.

As used herein, the term “nucleocapsid (N) protein” plays an important role in the viral packaging, viral nucleation and viral RNA synthesis of coronaviruses. As one of the structural proteins of coronaviruses, the nucleocapsid protein is a protein that performs various functions and plays an important role in the viral life cycle, and thus, is a good target capable of suppressing or detecting coronaviruses.

In the present invention, the compound may be characterized by suppressing coronavirus virion production, but is not limited thereto.

As used herein, the term “virion” refers to a virus particle in its complete form consisting of a capsid and nucleic acid. In general, the term “virion” refers to an infectious virus particle. Therefore, the production of the virion is associated with the smoothness of viral replication.

The pharmaceutical composition of the present invention may further comprise an appropriate carrier, excipient, and diluent, which are typically used to prepare a pharmaceutical composition. The excipient may be, for example, one or more selected from the group consisting of a diluent, a binder, a disintegrant, a lubricant, an adsorbent, a moisturizer, a film-coating material, and a controlled release additive.

The pharmaceutical composition according to the present invention may be used by being formulated into the form of a powder, a granule, a sustained-release granule, an enteric granule, a liquid, a collyrium, an elixir, an emulsion, a suspension, a spirit, a troche, aromatic water, a limonade, a tablet, a sustained-release tablet, an enteric tablet, a sublingual tablet, a hard capsule, a soft capsule, a sustained-release capsule, an enteric capsule, a pill, a tincture, a soft extract agent, a dry extract agent, a fluid extract agent, an injection, a capsule, a perfusate, an external preparation such as a plaster, a lotion, a paste, a spray, an inhalant, a patch, a sterilized injection solution, or an aerosol, and the external preparation may have a formulation such as a cream, a gel, a patch, a spray, an ointment, a plaster, a lotion, a liniment, a paste or a cataplasma.

Examples of a carrier, an excipient or a diluent which may be comprised in the composition according to the present invention comprise lactose, dextrose, sucrose, an oligosaccharide, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When the pharmaceutical composition is prepared, the pharmaceutical composition is prepared using a diluent or excipient, such as a filler, an extender, a binder, a wetting agent, a disintegrant, and a surfactant, which are commonly used.

As an additive of the tablet, powder, granule, capsule, pill, and troche according to the present invention, it is possible to use an excipient such as corn starch, potato starch, wheat starch, lactose, sucrose, glucose, fructose, D-mannitol, precipitated calcium carbonate, synthetic aluminum silicate, calcium monohydrogen phosphate, calcium sulfate, sodium chloride, sodium hydrogen carbonate, purified lanolin, microcrystalline cellulose, dextrin, sodium alginate, methyl cellulose, carboxymethyl cellulose sodium, kaolin, urea, colloidal silica gel, hydroxypropyl starch, hydroxypropyl methylcellulose (HPMC) 1928, HPMC 2208, HPMC 2906, HPMC 2910, propylene glycol, casein, calcium lactate, and Primojel; and a binder such as gelatin, arabic gum, ethanol, agar powder, cellulose acetate phthalate, carboxymethyl cellulose, carboxymethyl cellulose calcium, glucose, purified water, sodium caseinate, glycerin, stearic acid, carboxymethyl cellulose sodium, methylcellulose sodium, methylcellulose, microcrystalline cellulose, dextrin, hydroxycellulose, hydroxypropyl starch, hydroxymethyl cellulose, purified shellac, starch, hydroxypropyl cellulose, hydroxypropyl methyl cellulose, polyvinyl alcohol, and polyvinylpyrrolidone, and it is possible to use a disintegrant such as hydroxypropyl methyl cellulose, corn starch, agar powder, methyl cellulose, bentonite, hydroxypropyl starch, carboxymethyl cellulose sodium, sodium alginate, carboxymethyl cellulose calcium, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, arabic gum, amylopectin, pectin, sodium polyphosphate, ethyl cellulose, sucrose, magnesium aluminum silicate, a D-sorbitol solution, and light anhydrous silicic acid; and a lubricant such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol 4000, polyethylene glycol 6000, liquid paraffin, hydrogenated soybean oil (Lubriwax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid.

As an additive for liquid formulation according to the present invention, it is possible to use water, diluted hydrochloric acid, diluted sulfuric acid, sodium citrate, sucrose monostearate, polyoxyethylene sorbitol fatty acid esters (Tween esters), polyoxyethylene monoalkyl ether, lanolin ether, lanolin esters, acetic acid, hydrochloric acid, aqueous ammonia, ammonium carbonate, potassium hydroxide, sodium hydroxide, prolamin, polyvinyl pyrrolidone, ethyl cellulose, carboxymethyl cellulose sodium, and the like.

In a syrup according to the present invention, a solution of sucrose, other sugars or sweeteners, and the like may be used, and a fragrance, a colorant, a preservative, a stabilizer, a suspending agent, an emulsifier, a thickener, and the like may be used, if necessary.

Purified water may be used for the emulsion according to the present invention, and an emulsifier, a preservative, a stabilizer, a fragrance, and the like may be used, if necessary.

In the suspending agent according to the present invention, a suspending agent such as acacia, tragacanth, methyl cellulose, carboxymethyl cellulose, carboxymethyl cellulose sodium, microcrystalline cellulose, sodium alginate, hydroxypropyl methyl cellulose, HPMC 1828, HPMC 2906, and HPMC 2910 may be used, and a surfactant, a preservative, stabilizers, a colorant, and a fragrance may be used, if necessary.

The injection according to the present invention may comprise: a solvent such as distilled water for injection, 0.9% sodium chloride injection, Ringer's injection, dextrose injection, dextrose+sodium chloride injection, PEG, lactated Ringer's injection, ethanol, propylene glycol, non-volatile oil-sesame oil, cottonseed oil, peanut oil, corn oil, ethyl oleate, isopropyl myristate, and benzoic acid benzene; a solubilizing aid such as sodium benzoate, sodium salicylate, sodium acetate, urea, urethane, monoethyl acetamide, butazolidin, propylene glycol, Tweens, nijungtinateamide, hexamine, and dimethylacetamide; a buffer such as a weak acid and a salt thereof (acetic acid and sodium acetate), a weak base and a salt thereof (ammonia and ammonium acetate), an organic compound, a protein, albumin, peptone, and gums; an isotonic agent such as sodium chloride; a stabilizer such as sodium bisulfite (NaHSO₃), carbon dioxide gas, sodium metabisulfite (Na₂S₂O₅), sodium sulfite (Na₂SO₃), nitrogen gas (N₂), and ethylenediaminetetraacetic acid; an antioxidant such as 0.1% sodium bisulfide, sodium formaldehydesulfoxylate, thiourea, disodium ethylenediaminetetraacetate, and acetone sodium bisulfite; an analgesic such as benzyl alcohol, chlorobutanol, procaine hydrochloride, glucose, and calcium gluconate; and a suspending agent such as carboxymethyl cellulose sodium, sodium alginate, Tween 80, and aluminum monostearate.

In a suppository according to the present invention, it is possible to use a base such as cacao butter, lanolin, Witepsol, polyethylene glycol, glycerogelatin, methylcellulose, carboxymethyl cellulose, a mixture of stearic acid and oleic acid, Subanal, cottonseed oil, peanut oil, palm oil, cacao butter+cholesterol, lecithin, ranetwax, glycerol monostearate, Tween or Span, Imhausen, monolen (propylene glycol monostearate), glycerin, Adeps solidus, Buytyrum Tego-G, Cebes Pharma 16, hexalide base 95, Cotomar, Hydroxote SP, S-70-XXA, S-70-XX75 (S-70-XX95), Hydrokote 25, Hydrokote 711, idropostal, Massa estrarium (A, AS, B, C, D, E, I, T), Massa-MF, Masupol, Masupol-15, Neosupostal-ene, Paramound-B, Suposhiro (OSI, OSIX, A, B, C, D, H, L), suppository base IV types (AB, B, A, BC, BBG, E, BGF, C, D, 299), Supostal (N, Es), Wecobee (W, R, S, M,Fs), and tegester triglyceride base (TG-95, MA, 57).

A solid formulation for oral administration comprises a tablet, a pill, a powder, a granule, a capsule, and the like, and the solid formulation is prepared by mixing at least one excipient, for example, starch, calcium carbonate, sucrose or lactose, gelatin, and the like with an extract. Further, in addition to a simple excipient, lubricants such as magnesium stearate and talc are also used.

A liquid formulation for oral administration corresponds to a suspension, a liquid for internal use, an emulsion, a syrup, and the like, and the liquid formulation may comprise, in addition to water and liquid paraffin which are simple commonly used diluents, various excipients, for example, a wetting agent, a sweetener, a fragrance, a preservative, and the like. Examples of a formulation for parenteral administration comprise an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like.

The pharmaceutical composition according to the present invention is administered in a pharmaceutically effective amount. In the present invention, “pharmaceutically effective amount” means an amount sufficient to treat diseases at a reasonable benefit/risk ratio applicable to medical treatment, and an effective dosage level may be determined according to factors comprising the type of disease of patients, the severity of disease, the activity of drugs, sensitivity to drugs, administration time, administration route, excretion rate, treatment period, and simultaneously used drugs, and other factors well known in the medical field.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by a person with ordinary skill in the art to which the present invention pertains.

The pharmaceutical composition of the present invention may be administered to a subject in need via various routes. All methods of administration may be expected, but the pharmaceutical composition may be administered by, for example, oral administration, subcutaneous injection, peritoneal administration, intravenous injection, intramuscular injection, paraspinal space (intradural) injection, sublingual administration, buccal administration, intrarectal insertion, intravaginal injection, ocular administration, ear administration, nasal administration, inhalation, spray via the mouth or nose, skin administration, transdermal administration, and the like.

The pharmaceutical composition of the present invention is determined by the type of drug that is an active ingredient, as well as various related factors such as the disease to be treated, the route of administration, the age, sex, and body weight of a patient, and the severity of the disease.

As used herein, the “subject” refers to a subject in need of treatment of a disease, and more specifically, refers to a mammal such as a human or a non-human primate, a mouse, a rat, a dog, a cat, a horse, and a cow.

The “administration” as used herein refers to the provision of a predetermined composition of the present invention to a subject in need thereof by any suitable method. As used herein, the “prevention” refers to all actions that suppress or delay the onset of a target disease, and the “treatment” refers to all actions that ameliorate or beneficially change a target disease and the resulting metabolic abnormalities by administration of the pharmaceutical composition according to the present invention, and the “amelioration” refers to all actions that reduce a target disease and associated parameters, for example, the severity of symptoms, by administration of the composition according to the present invention.

Further, the present invention provides a food composition for preventing or ameliorating coronavirus infectious diseases, comprising, as an active ingredient, a compound that binds to G4 structures of coronaviruses or a sitologically acceptable salt thereof.

When the compound that binds to G4 structures of coronaviruses of the present invention is used as a food additive, the compound may be added as it is or used with another food or other food ingredients, and may be appropriately used according to a typical method. The amount of active ingredient mixed may be suitably determined according to the purpose of use (prevention, health or therapeutic treatment). In general, when a food or beverage is prepared, the compound of the present invention may be added in an amount of 15 wt % or less, or 10 wt % or less based on the raw materials. However, in the case of long-term intake for the purpose of health and hygiene or for the purpose of controlling health, the amount may be equal to or less than the above range, and the effective ingredient may be used in an amount equal to or more than the above range due to no problem in terms of safety.

The type of food is not particularly limited. Examples of food to which the material may be added comprise meats, sausage, bread, chocolate, candies, snacks, confectioneries, pizza, instant noodles, other noodles, gums, dairy products comprising ice creams, various soups, beverages, tea, drinks, alcoholic beverages, vitamin complexes, and the like, and comprise all health functional foods in a typical sense.

The health beverage composition according to the present invention may comprise various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may comprise various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may comprise flesh for preparing natural fruit juice, fruit juice drinks, and vegetable drinks. These ingredients may be used either alone or in combinations thereof. The proportion of these additives is not very important, but is generally selected within a range of 0.01 to 0.20 part by weight per 100 parts by weight of the composition of the present invention.

The present invention provides a quasi-drug composition for preventing or suppressing coronavirus infectious diseases, comprising a compound that binds to G4 structures of coronaviruses as an active ingredient.

In the “quasi-drug composition for preventing or suppressing coronavirus infectious diseases” according to the present invention, the quasi-drug is a preparation that is used for sterilization, insecticides, and other similar uses for the purpose of preventing infectious diseases listed in Article 2, Paragraph 7, Item C of the Pharmaceutical Affairs Act, and may mean a repellent, an exterminator, an inhibitor, a control agent or an attractant insecticide of flies, mosquitoes and the like used for human or animal health.

A target to which the quasi-drug is applied comprises not only the subject, but also all living and non-living things, and is not limited thereto.

In addition, the quasi-drug may comprise a skin external preparation and a personal hygiene product. For example, the quasi-drug may be an antiseptic cleaner, shower foam, a mouthwash, a wet wipe, a detergent soap, a hand wash, or an ointment, but is not limited thereto.

When the quasi-drug composition according to the present invention is used as a quasi-drug additive, the composition may be added as it is or used with another quasi-drug or other quasi-drug ingredients, and may be appropriately used according to a typical method. A mixing amount of the active ingredient may be determined appropriately according to the purpose of use.

The quasi-drug composition of the present invention may be prepared in the form of, for example, a general emulsion formulation and a general solubilized formulation. The quasi-drug composition according to the present invention may have, for example, a formulation such as an emulsion such as a lotion, a cream, an ointment, a spray, an oil gel, a gel, an oil, an aerosol, and a fogging agent, but any material can be used without limitation as long as it exhibits the pest control-inducing effect of the present invention. In addition, the quasi-drug composition may be used by appropriately blending oil, water, a surfactant, a moisturizing agent, a lower alcohol having 1 to 4 carbon atoms, a thickener, a chelating agent, a pigment, a preservative, a perfume, or the like, which is generally blended in quasi-drug compositions, with each formulation, if necessary.

The present invention provides an antiviral composition against coronaviruses, comprising a compound that binds to G4 structures of coronaviruses as an active ingredient.

In the present invention, the “antiviral” refers to weakening or eliminating the action of viruses that have invaded the body by suppressing the proliferation of viruses in the body, and more specifically, refers to suppressing the proliferation of viruses by inhibiting the process of viral nucleic acid synthesis, gene expression, or viral replication, and is intended for coronaviruses in the present invention.

A target to which the composition is applied comprises the above-described individual, but also all living and non-living things, and is not limited thereto.

In the present invention, the antiviral composition may have antiviral activity against human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, Middle East respiratory syndrome coronavirus (MERS-COV), and severe acute respiratory syndrome coronavirus 2 (SARS-COV-2 or 2019 novel coronavirus or 2019-nCOV).

With respect to the terms used in the present invention, general terms currently and widely used are selected in consideration of function in the present invention, but the terms may vary according to an intention of a technician skilled in the art, a precedent, an advent of a new technology, and the like. Further, in specific cases, there is also a term arbitrarily chosen by the applicant, and in this case, the meanings thereof will be described in detail in the corresponding part of the Detailed Description of the present invention. Accordingly, the term used in the present invention should not be defined merely as a simple name of the term, but should be defined based on the meaning of the term and overall content of the present invention.

Hereinafter, preferred examples for helping with understanding of the present invention will be suggested. However, the following examples are provided only so that the present invention may be more easily understood, and the content of the present invention is not limited by the following examples.

### Experimental Methods

### 1. Construction of Oligonucleotides

A 100 μM stock solution was prepared by dissolving RNA oligonucleotides (Bioneer, Korea) in 200 μl of nuclease-free water according to the manufacturer's protocol. RNA G4 oligonucleotides used in the present invention are shown in the following Table 1.

TABLE 1

| SEQ | | | G4 |
|---|---|---|---|
| ID | Genome | | sequence |
| NO: | position | G4 sequence | length |
| 1 | 353 | GGCUUUGGAGACUCC | 25 |
| | | GUGGAGGAGG | |
| 2 | 644 | GGUAAUAAAGGAGCU | 20 |
| | | GGUGG | |
| 3 | 1463 | GGUGGUCGCACUAUG | 26 |
| | | CCUUUGGAGG | |
| 4 | 1574 | GGUGUUGUUGGAGAA | 26 |
| | | GGUUCCGAAGG | |
| 5 | 2714 | GGCGGUGCACCAACA | 29 |
| | | AAGGUUACUUUUGG | |
| 6 | 3467 | GGAGGAGGUGUUGCA | 17 |
| | | GG | |
| 7 | 4162 | GGUUAUACCUACUAA | 27 |
| | | AAAGGCUGGUGG | |
| 8 | 4261 | GGGUUUAAAUGGUUA | 29 |
| | | CACUGUAGAGGAGG | |
| 9 | 8687 | GGAUACAAGCUAUUG | 23 |
| | | AUGGUGG | |
| 10 | 10267 | GGCUGGUAAUGUUCA | 30 |
| | | ACUCAGGGUUAUUGG | |
| 11 | 13385 | GGUAUGUGGAAAGGU | 20 |
| | | UAUGG | |
| 12 | 14947 | GGUUUUCCAUUUAAU | 28 |
| | | AAAUGGGGUAAGG | |
| 13 | 15208 | GGAACAAGCAAAUUC | 29 |
| | | UAUGGUGGUUGG | |
| 14 | 15448 | GGCGUUCACUAUAUG | 29 |
| | | UUAAACCAGGUGG | |
| 15 | 15290 | GGAUUGGCUUCGAUG | 23 |
| | | UCGAGGGG | |
| 16 | 22316 | GGUGAUUCUUCUUCA | 29 |
| | | GGUUGGACAGCUGG | |
| 17 | 24215 | GGUUGGACCUUUGGU | 20 |
| | | GCAGG | |
| 18 | 24268 | GGCUUAUAGGUUUAA | 24 |
| | | UGGUAUUGG | |
| 19 | 25197 | GGCCAUGGUACAUUU | 22 |
| | | GGCUAGG | |
| 20 | 25951 | GGUGGUUAUACUGAA | 29 |
| | | AAAUGGGAAUCUGG | |
| 21 | 26746 | GGAUCACCGGUGGAA | 30 |
| | | UUGCUAUCGCAAUGG | |
| 22 | 26761 | GGCUUCUACGCAGAA | 29 |
| | | GGGAGCAGAGGCGG | |
| 23 | 28903 | GGCUGGCAAUGGCGG | 15 |
| 24 | 29122 | GGAAAUUUUGGGGAC | 19 |
| | | CAGG | |
| 25 | 29234 | GGCAUGGAAGUCACA | 30 |
| | | CCUUCGGAACGUGG | |

2. Measurement of Circular Dichroism (CD) Spectra 15 μM RNA oligonucleotides were annealed in a buffer comprising 10 mM Tris-HCl (pH 7.5) and 100 mM KCl in a thermoblock (denaturation at 95° C. for 10 minutes followed by cooling to 4° C. at 0.5° C./min). Samples were stored at 4° C. overnight before performing experiments. CD spectra were measured between 220 and 320 nm wavelengths with a scan rate of 100 nm/min, a data pitch of 1 nm and a bandwidth of 1 nm using a 1 mm quartz cuvette at 20° C. In addition, each spectrum was measured three times and the average is shown. CD experiments were performed using a Jasco J-810 CD spectropolarimeter fitted with a Jasco CDC-426F Peltier temperature controller.

### 3. CD Thermal Melting Analysis

For melting, the samples were heated at 25 to 95° C. at 2° C./min with a data pitch of 0.5° C. CD melting analyses were performed with and without G4-binding compounds. The ratio of RNA: G4-binding compound was set to a molar ratio of 1:2. 15 μM RNA G4 was treated with 30 μM G4-binding compound and incubated at room temperature in a buffer comprising 10 mM Tris-HCl (pH 7.5) and 100 mM KCl for 30 minutes. Ellipticity was measured at 265 nm. Thereafter, data were normalized and plotted in OriginPro 2017 to calculate Tm values.

### 4. Cell Culture

Human embryonic kidney (HEK293T) cells and Vero cells were maintained in high-glucose Dulbecco's Modified Eagle's Medium (DMEM) supplemented with 10% fetal bovine serum (FBS) and 1% penicillin-streptomycin, and cultured in a humidified incubator at 37° C. and 5% CO₂.

5. Construction of peYFP-N1 Reporter Vector for In Vitro Expression

A pEYFP-N1 reporter (Promega) vector was generated by cloning an eYFP coding region and a target gene (WT-G4-353, 644, 3467) in a frame in order to encode an eYFP fusion protein. Primers used for PCR amplification cloning are shown in the following Table 2. Target gene inserts for WTG4-353, 644, 3467 were synthesized by PUC-GW-Amp (donor plasmid) (Cosmogenetech), subcloned between the Xho1/BamH1 restriction sites of a recipient plasmid pEYFP-N₁ vector, and then transformed into competent E. coli DH5α cells. The named recombinant plasmids (N1-WT-G4-353, 644 and 3467) were each confirmed by sequencing.

TABLE 2

| | SEQ | | Ref- |
|---|---|---|---|
| Name | ID NO: | Primer sequence | erence |
| WT G4-353 | 26 | TATAGCTCGAGATGGAGAGCCTTG | This |
| (Fwd) | | TCC | study |
| WT G4-353 | 27 | TATACGGATCCACATGACCATGAG | This |
| (Rev) | | GTGC | study |
| WT G4-644 | 26 | TATACCTCGAGATGGTTGAGCTGG | This |
| (Fwd) | | TAGC | study |
| WT G4-644 | 29 | TATACGGATCCCCGTTAAGCTCAC | This |
| (Rev) | | GC | study |
| WT G4-3467 | 30 | GATAACTCGAGATGGAACTTACAC | This |
| (Fwd) | | CAGTTGTTCAGAC | study |
| WT G4-3467 | 31 | AATATAGGATCCGGAATCTCAGCG | This |
| (Rev) | | ATCTTTTGTTC | study |
| Mut G4-353 | 32 | CGCGACGTGCTCGTACGTGACTTT | This |
| (Fwd) | | GAAGACTCCGTAGAAGAAGTC | study |
| Mut G4-353 | 33 | GACTTCTTCTACGGAGTCTTCAAA | This |
| (Rev) | | GTCACGTACGAGCACGTCGCG | study |
| Mut G4-544 | 34 | CGGTAATAAAAGAGCTAGTAGCCA | This |
| (Fwd) | | TAGTTACGGCGCCG | study |
| Mut G4-544 | 35 | CGGCGCCGTAACTATGGCTACTAG | This |
| (Rev) | | CTCTTTTATTACCG | study |
| Mut G4-3467 | 36 | CATGGAAGAAGTGTTGCAAGAGCC | This |
| (Fwd) | | TTAAATAAGGCTAC | study |
| Mut G4-3467 | 37 | GTAGCCTTATTTAAGGCTCTTGCA | This |
| (Rev) | | ACACTTCTTCCATG | study |
| T7-G4-353 | 38 | AGTAATACGACTCACTATAGGGAT | This |
| (Fwd) | | GGAGAGCCTTGTCC | study |
| T7-G4-644 | 39 | AGTAATACGACTCACTATAGGGAT | This |
| (Fwd) | | GGTTGAGCTGGTAGCAG | study |
| T7-G4-3467 | 40 | AGTAATACGACTCACTATAGGGAT | This |
| (Fwd) | | GGAACTTACACCAGTTGTTC | study |
| T7-EYFP | 41 | TTACTTGTACAGCTCGTCCATG | This |
| | | | study |

### 6. Site-Directed Mutagenesis

To introduce site-directed mutations that interfere with G4 formation in G4-353, G4-644, and G4-3467, PCR reactions were performed according to the QuikChange site-directed mutagenesis protocol (Stratagene). Primers used for mutagenesis are shown in Table 2 above. After mutagenesis, a wild-type vector backbone was digested with Dpn1 at 37° C. overnight and transformed into DH5α competent cells. The obtained positive clones were confirmed by sequencing. The named mutant G4 plasmids (N₁-Mut-G4-353, 644 and 3467) were confirmed by sequencing.

7. Native Polyacrylamide Gel Electrophoresis Native polyacrylamide gel electrophoresis was performed on 15% polyacrylamide gels in 1×TBE buffer comprising 50 mM KCl at 80 V and 4° C. for 3 hours. The gels were stained with SYBR Gold nucleic acid dye and observed using a Bio-Rad Gel Doc UV-transilluminator.

### 8. Cell Viability Analysis

Cytotoxicity was analyzed using an EZ Cytox assay kit (DogenBio, Korea). HEK293T and Vero cells were put into a 96-well plate at a density of 2×10⁴ cells/well in a medium volume of 100 μl/well and cultured at 37° C. for 24 hours. After 24 hours, the cells were treated with the G4-binding compound at various concentrations (0.1, 0.5, 1, 2.5, 5.5, 7.5, 10, 25, 50, and 75 μM) and cultured at 37° C. for 24 hours or 48 hours. Cell viability (%) was calculated by measuring the absorbance value at 455 nm (Luminescence multi-mode microplate reader Synergy™ Neo2) and using the following Equation 1. All experiments were performed in triplicate, and cells untreated with the compound were used as a control. DMEM with the EZ Cytox reagent was used as a blank.

$$Cell viability ( \% ) = [ ( {A}_{455 ( Test sample )} - {A}_{455 ( Blank )} ) / ( {A}_{455 ( Control sample )} - {A}_{455 ( Blank )} ) ] * 100 \qquad [ Equation 1 ]$$

### 9. Cell Transfection

Transfection was performed on HEK293T and Vero cells according to the manufacturer's protocol (TurboFect™ Transfection Reagent, Invitrogen, USA). 1.5×10⁵ cells or 7.5×10⁴ cells were put into 2 ml of a DMEM medium in a 6-well plate, respectively, and cultured at 37° C. After 24 hours, the cells were transfected according to the manufacturer's instructions. After 10 hours, the cells were treated with a G4-binding compound by replacing the medium with a medium comprising or not comprising the G4-binding compound. Thereafter, the cells were cultured for 24 or 48 hours, and harvested for further experiments.

### 10. In-Vitro Transcription/Translation (IVT) Analysis

WT-G4 and Mut-G4 sequences were PCR amplified with specific primers designed as T7 promoter sites. The PCR product has a length of 1 Kbp and comprised the T7 promoter site and an eGFP gene. Primers used for IVT are shown in Table 2 above. PCR products were transcribed and translated using a TNT Quick Coupled Transcription/Translation System (Promega), and a PCR-DNA template (0.5 μg) was translated in 50 μl of a reaction mixture comprising 1 mM methionine at 30° C. for 90 minutes. Translated products were dissolved using 12% SDS-PAGE and analyzed by western blot.

### 11. Western Blot

Cells were lysed in 1X-RIPA buffer. Isolated proteins were transferred to a polyvinylidene fluoride (PVDF) membrane (Immobilon-PVDF membrane for western blotting, Merck KGaA, Darmstadt, Germany) using a Bio-Rad semi-dry transfer system. Non-specific protein binding was blocked with 5% non-fat milk in Tris-buffered saline comprising 0.05% Tween-20 (TBS-T) buffer for 1 hour. Thereafter, the membrane was washed with TBST buffer and probed with a mouse monoclonal anti-EYFP antibody (MAB8759, Abnova) which is a primary antibody diluted 1:5000 at 4° C. for 1 hour. Thereafter, protein detection was performed using a secondary goat-anti-mouse antibody (SC-2031, Santa Cruz Biotechnology, South Korea) diluted 1:5000 to which HRP was conjugated at room temperature for 1 hour. Bands were detected using an enhanced chemiluminescence (ECL) western blot substrate by a luminescence image analyzer LAS3000. EYFP and intensity were normalized to the housekeeping gene GAPDH band intensity. The band intensity was quantified using ImageJ.

### 12. Statistical Analysis

Data from Experiments 10 and 11 was analyzed using GraphPad Prism software version 8.3.0. A one-way ANOVA test was used to determine significant differences between drug-untreated samples and drug-treated samples by EYFP translational inhibition. Data was expressed as mean±SEM or mean±SD. * p≤0.05, ** p≤0.01, *** p<0.001, **** p<0.0001.

### 13. SARS-COV-2 Dose Response Curve (DRC) Analysis-N Protein Expression Relationship

### 13-1. Viruses and Cell Lines

SARS-COV-2 was provided by the Korea Disease Control and Prevention Agency (KDCA) and Vero cells were obtained from ATCC (ATCC-CCL81).

### 13-2. Reagents

Chloroquine, lopinavir, and remdesivir used as reference compounds were purchased from Sigma-Aldrich, SelleckChem, and MedChemExpress, respectively. A primary antibody specific for the anti-SARS-COV-2 nucleocapsid (N) protein was purchased from Sino Biological, and a secondary antibody Alexa Fluor 488 goat anti-rabbit IgG and Hoechst 33342 were purchased from Molecular Probes.

### 13-3. Dose Response Curve (DRC) Analysis by Immunofluorescence

Vero cells were seeded in a 384-well plate at a cell density of 1.2×10⁴ cells/well in a DMEM supplemented with 2% FBS. After 24 hours, the cells were treated with G4-binding compounds (NMM, BRACO19, PDS and PhenDC3) prepared at 10 points by 2-fold serial dilutions in DMSO at concentrations ranging from 0.08 to 50 μM. As a positive control, chloroquine diphosphate (C6628, Sigma-Aldrich, St. Louis, MO), lopinavir (S1380, SelleckChem, Houston, TX) and remdesivir (HY-104077, MedChemExpress, Monmouth Junction, NJ), whose antiviral activity is well known, were used. After about 1 hour, cells were infected with SARS-COV-2 (0.0125 MOI) in a BSL3 facility and cultured at 37° C. for 24 hours. Thereafter, the cells were fixed with 4% paraformaldehyde (PFA) and then permeabilized. Thereafter, the cells were stained by being treated with an anti-SARS-COV-2 nucleocapsid (N) protein primary antibody and treated with an Alexa Fluor 488 goat anti-rabbit IgG secondary antibody and Hoechst 33342. Fluorescence images of infected cells were obtained using Operetta (Perkin Elmer), which is a high capacity image analysis instrument.

### 13-4. Image Analysis

Acquired images were analyzed using Columbus software. The total number of cells per well was calculated as the number of nuclei stained with Hoechst, and the number of infected cells was calculated as the number of cells expressing the viral N protein. An infection ratio was calculated as (number of cells expressing N protein)/(total number of cells). The infection ratio per well was normalized to the average infection ratio of wells comprising uninfected cells (mock) and the average infection ratio of wells comprising infected cells treated with 0.5% DMSO (v/v) in the same plate. The reaction curve and IC₅₀ and CC₅₀ values depending on the compound concentration were derived using the following Equation 2 of XLFit 4 (IDBS) software.

$$Y = Bottom + ( Top - Bottom ) / ( 1 + {( {IC}_{50} / X )}^{Hillslope} ) \qquad [ Equation 2 ]$$

All IC₅₀ and CC₅₀ values were calculated from fitted dose response curves obtained in duplicate experiments, and selectivity index (SI) values were calculated as CC₅₀/IC₅₀.

### 14. SARS-COV-2 Dose Response Curve (DRC) Analysis-Virion Production Relationship

Vero cells were seeded in a 12-well plate at a cell density of 2×10⁵ cells/well. After 24 hours, the cells were treated with G4-binding compounds (PDS, PhenDC3 and NMM) prepared at 10 points by 2-fold serial dilutions for 1 hour in a BSL3 facility at concentrations ranging from 0.08 to 50 μM. The cells were washed once with PBS and then infected with SARS-COV-2 (0.01 MOI) for 1 hour. After the virus inoculum was removed and the cells were washed once with PBS, the medium was replaced with a 2% FBS DMM medium comprising the same G4-binding compound as above at each concentration and incubated for 24 hours or 48 hours. DMSO and distilled water vehicles were also added as controls for each compound. 24 or 48 hours after infection, viral supernatants were collected and total RNA was extracted by automated nucleic acid purification using a PROMEGA Maxwell RSC48 instrument. RT-qPCR was performed using primers specific for SARS-COV-2RdRp in duplicate per sample using a THERMO QuantStudio™ 3 system. Viral genomic RNA transcript levels were analyzed by the ΔΔCt method and relative inhibition of infection was calculated by normalizing groups treated with the G4-binding compound to the DMSO or distilled water control. The IC₅₀ for each compound was derived utilizing the following Equation 3 in Graphpad Prism 5.0 software.

$$Y = Bottom + ( Top - Bottom ) / ( 1 + {( {IC}_{50} / X )}^{Hillslope} ) \qquad [ Equation 3 ]$$

### 15. HCoV-OC43 Dose Response Curve (DRC) Analysis-Virion Production Relationship

HCT-8 cells, which are a human colorectal cancer cell line, were seeded in a 12-well plate at a cell density of 2×10⁵ cells/well. After 24 hours, the cells were treated with G4-binding compounds (CX3543, NMM, TMPyP4 and TMPyP2) prepared at 10 points by 2-fold serial dilutions for 1 hour at concentrations ranging from 0.08 to 50 μM. The cells were washed once with PBS and then infected with HCoV-OC43 (0.05 MOI) for 1 hour. After the virus inoculum was removed and the cells were washed once with PBS, the medium was replaced with a 2% FBS DMM medium comprising the same G4-binding compound as above at each concentration and incubated for 72 hours. DMSO and distilled water vehicles were also added as controls for each compound. 72 hours after infection, viral supernatants were collected and the titer of the infectious virus was quantified by plaque assay.

In the plaque assay, Vero cells seeded in a 12-well plate one day before were inoculated in duplicate with 10-fold serially diluted virus supernatants in two wells, cultured for 1 hour, and then removed. A plaque assay overlay medium comprising 0.5% methylcellulose was added to the DMEM culture solution and incubated for 5 to 6 days. After 5 to 6 days of culture, the overlay medium was removed, and the cells were fixed and stained with a solution comprising a 2% crystal violet staining solution in 20% ethanol to count the number of plaques and measure the titer of the virus. In the viral titer of each sample, relative inhibition of infection was calculated by normalizing groups treated with the G4-binding compound to the DMSO or distilled water control. The IC₅₀ for each compound was derived utilizing Equation 3 above in Graphpad Prism 5.0 software.

For cytotoxicity in HCT-8 cells, the cells seeded in the 96-well plate were treated in duplicate with the G4-binding compound prepared at the same concentration as in the dose analysis experiment described above, and after 72 hours, each well was treated with 10 μl of 5 μg/ml 3-(4,5-dimethylthiazol-2-yl)-2,5-diphenyltetrazolium bromide (MTT) at 37° C. for 3 hours. After the treatment, the cells were washed once with PBS by removing the supernatant, and then 100 μl of DMSO was added, and the cells were additionally treated in the dark for 1 hour, and then analyzed by measuring the absorbance at a wavelength of 540 nm. For cytotoxicity of each compound at each concentration, relative cytotoxicity was calculated by normalizing groups treated with the G4-binding compound to the DMSO or distilled water controls.

### EXAMPLES

### Example 1. Confirmation of G4 of SARS-COV-2 Genome by Bioinformatics Analysis

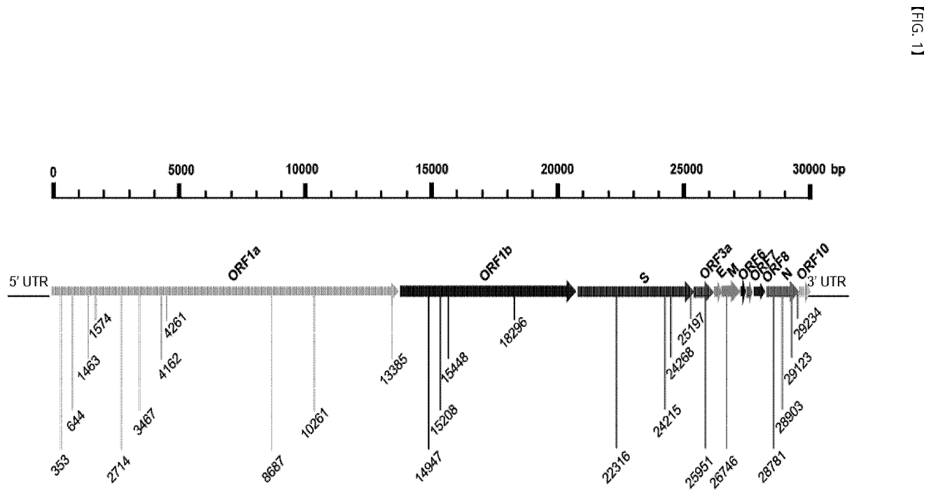

A QGRS mapper was applied to the SARS-COV-2 genome based on a maximum length of 30 bp, and 25 G4s with a minimum G-group length of 2 and a loop size ranging from 0 to 36 were identified. The results are illustrated in FIG. 1.

As illustrated in FIG. 1, G4 was located in the open reading frame regions of the ORF-lab, spike(S), ORF3a, membrane (M) and nucleocapsid (N) genes. Further, G4 appeared at a higher density in genomic regions encoding Nsps (encoded by ORF1 ab) and at a lower density in genomic regions encoding structural proteins. In addition, G4 with a G group length of 3 or 4 was not found in the genome of SARS-CoV-2.

### Example 2. Formation of Stable G4 Structure of G4 Predicted in SARS-CoV-2

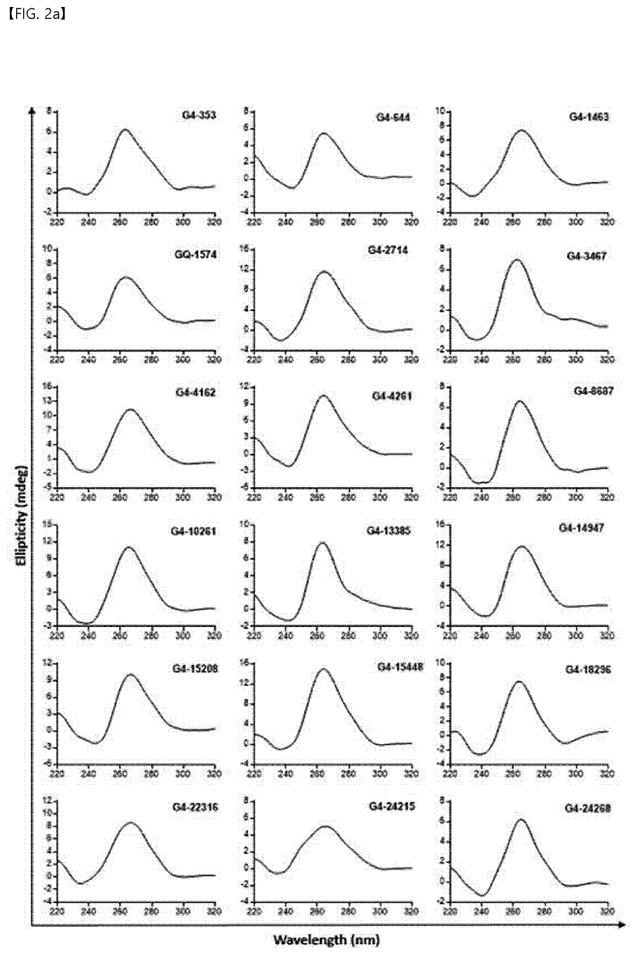

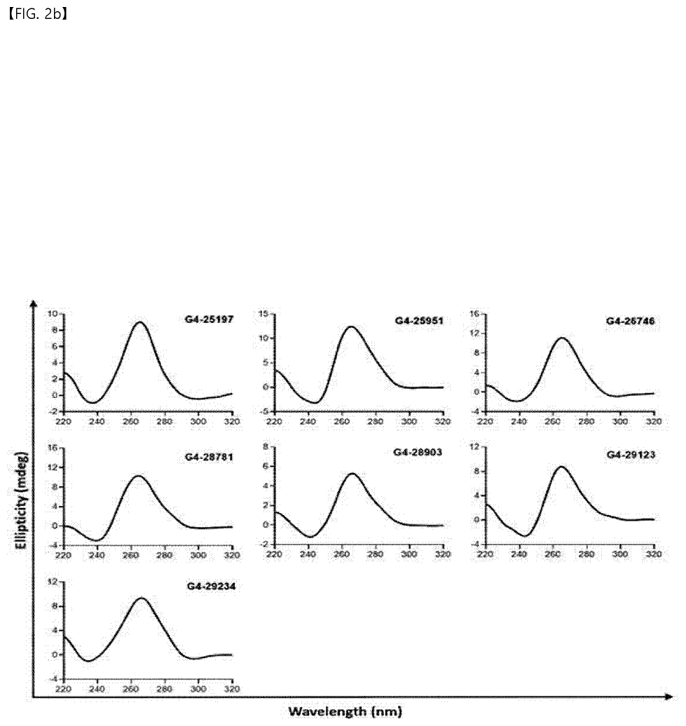

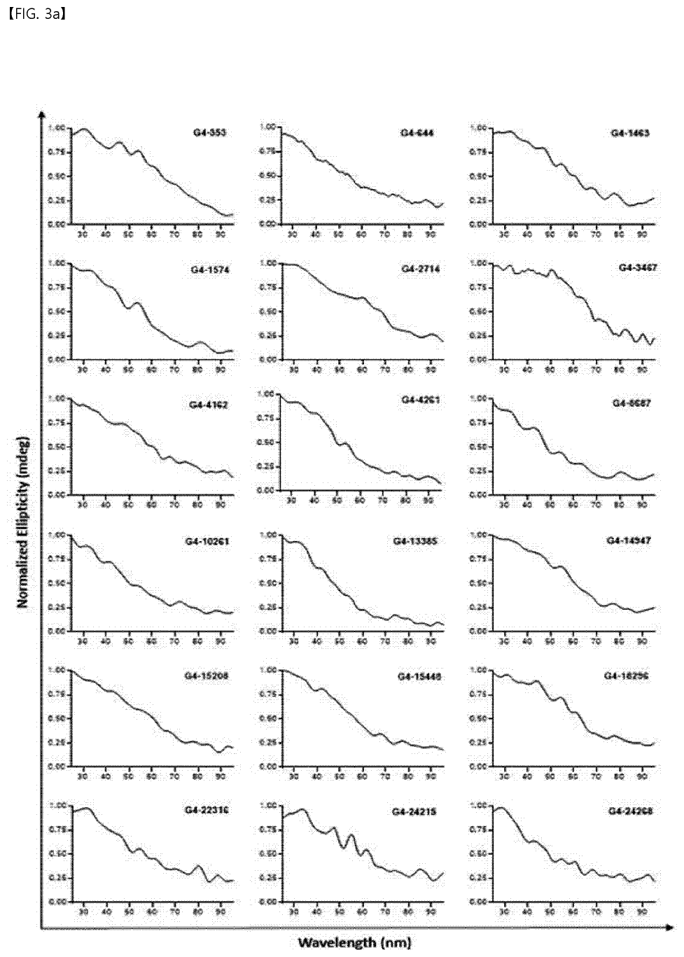

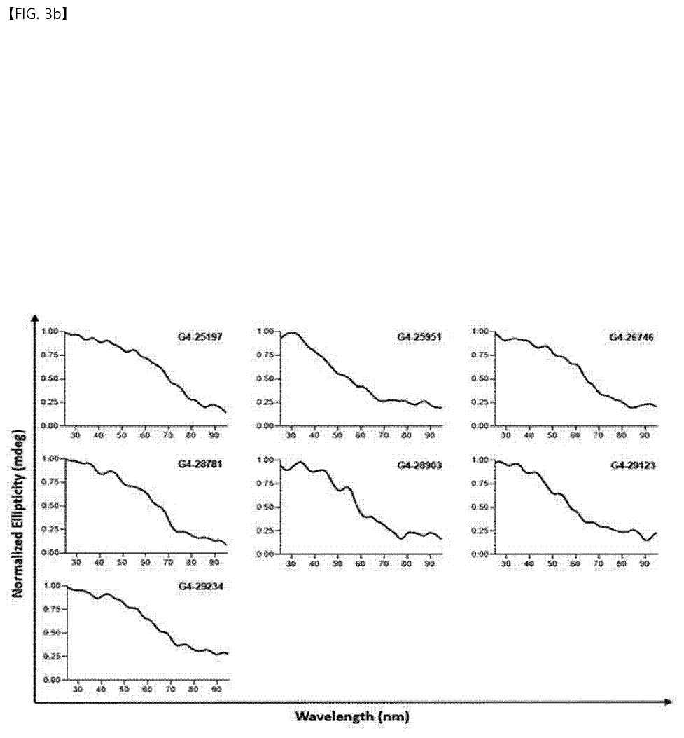

To confirm the formation and structural stability of G4s, biophysical studies were performed on all predicted G4s in SARS-COV-2 using RNA oligonucleotides corresponding to G4s. The formation of the G4 structure was confirmed by measuring the circular dichroism (CD) spectrum, and the structural stability of G4 was confirmed through the CD thermal melting analysis. The results are illustrated in FIGS. 2A and 2B and FIGS. 3A and 3B.

As illustrated in FIGS. 2A and 2B, the CD spectra of all SARS-COV-2G4 confirmed the G4 topology characterized by a strong positive peak at about 265 nm and a negative peak at about 240 nm.

Furthermore, SARS-COV-2 G4 exhibited a Tm in a range of 50 to 75° C., as illustrated in FIGS. 3A and 3B. This means that the G4 sequence predicted for SARS-COV-2 can fold into a stable G4 structure in vitro.

### Example 3. Instability of G4 According to Introduction of Site-Directed Mutations to Nsp1 and Nsp3

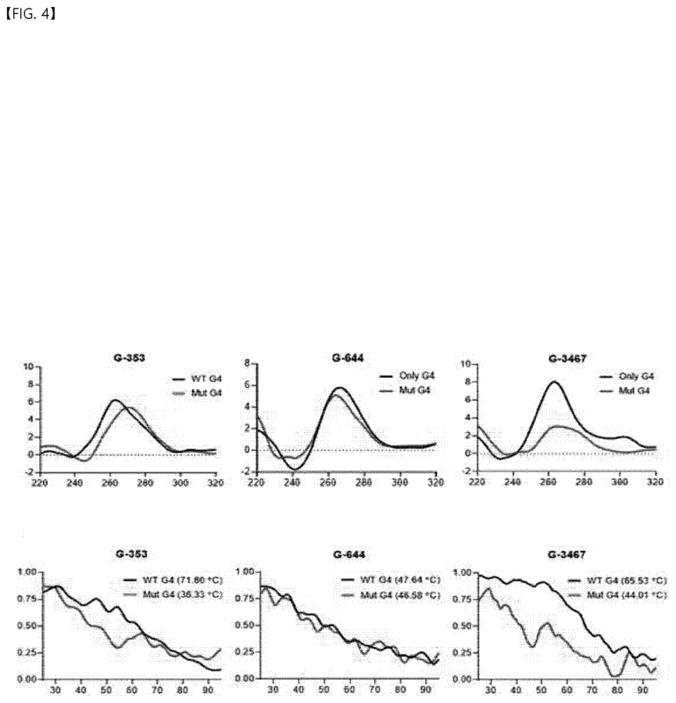

Since G4s (G4-353, G4-644, and G4-3467) located in Nsp1 and Nsp3 showed the highest ΔTm when treated with PDS and PhenDC3 as shown in the following Table 4, and have important biological functions, the G4s were selected as a mutation target. To visualize the effect of SARS-COV-2 G4 targeting on Nsp1 and Nsp3, guanine (G) highlighted in red in the WT-G4 sequence was mutated to adenine (A) to make Mut-G4 which interferes with the formation of G4. This is shown in the following Table 3. Thereafter, the CD spectra of WT and Mut G-353, G-644, and G-3467 were confirmed. The results are illustrated in FIG. 4.

As illustrated in FIG. 4, compared to the CD spectrum of WT-G4, Mut-G4 showed that the G4 topology was disrupted because the maximum value was shifted from about 260 nm to about 270 nm. Further, Mut-G4 exhibited a lower Tm value than WT-G4 in the CD thermal melting analysis. This implies the instability of G4 in Mut-G4.

TABLE 3

| | | WT-G4 | | Mut-G4 |
|---|---|---|---|---|
| Name | WT-GR sequences | Tm in ° C. | Mut-G4 sequences | Tm in ° C. |
| G-353 | GGCUUUGGAGACUCCGUGGAGG | 71.59 | GACUUUGAAGACUCCGUAGAAG | 36.22 |
| | AGG(SEQ ID NO: 1) | | AAG(SEQ ID NO: 42) | |
| G-644 | GGUAAUAAAGGAGCUGGUGG | 47.73 | GGUAAUAAAAGAGCUAGUAG | 46.64 |
| | (SEQ ID NO: 2) | | (SEQ ID NO: 43) | |
| G-3467 | GGAGGAGGUGUUGCAGG | 65.55 | GGAAGAAGUGUUGCAAG | 45.83 |
| | (SEQ ID NO: 6) | | (SEQ ID NO: 44) | |

### Example 4. SARS-COV-2 G4 Stability According to Treatment with G4-Binding Compound

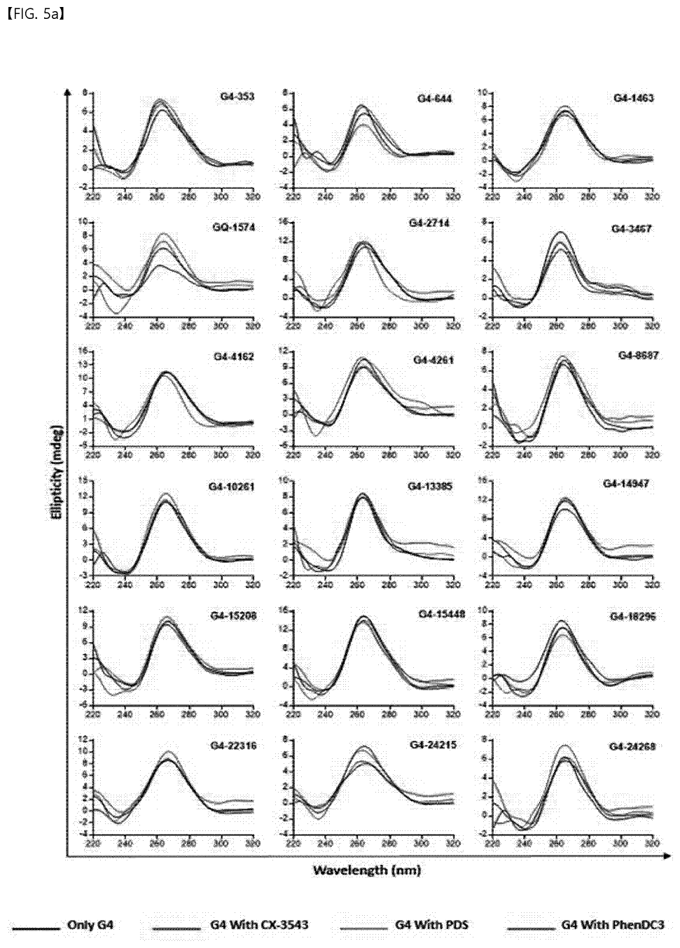

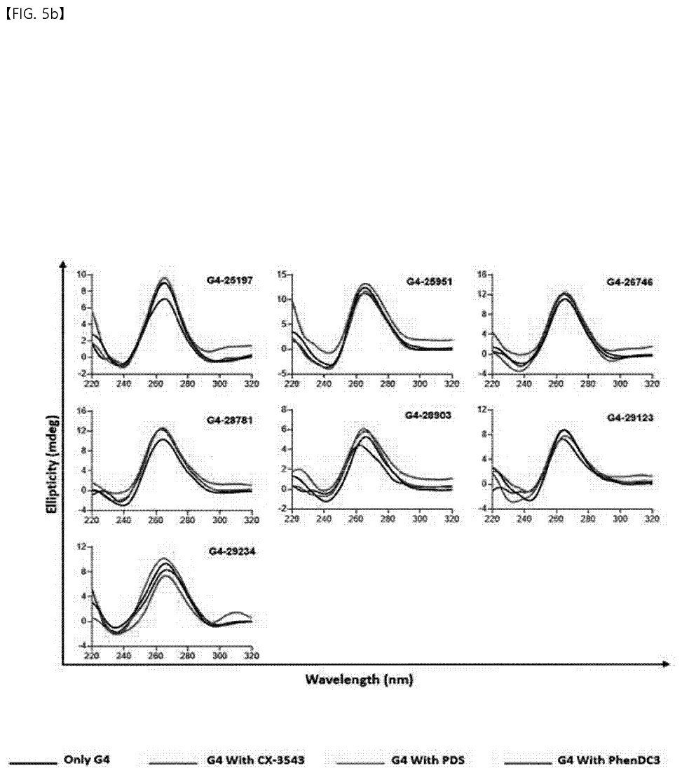

15 μM RNA oligonucleotides were annealed in 10 mM Tris-HCl (pH 7.5) and 100 mM KCl. Thereafter, the RNA oligonucleotides were treated with 30 μM compound and incubated. Next, 25 G4s present in SARS-COV2 were confirmed through spectra features appearing through a circular dichroism analyzer. Each spectrum is the average of triplicate measurements. The results are illustrated in FIGS. 5A and 5B.

As illustrated in FIGS. 5A and 5B, during treatment with the G4-binding compound, the CD spectrum had a positive peak at about 265 nm and a negative peak at about 240 nm to show no considerable change in the spectrum of Example 2. This means that G4-binding compounds do not alter the G4 topology.

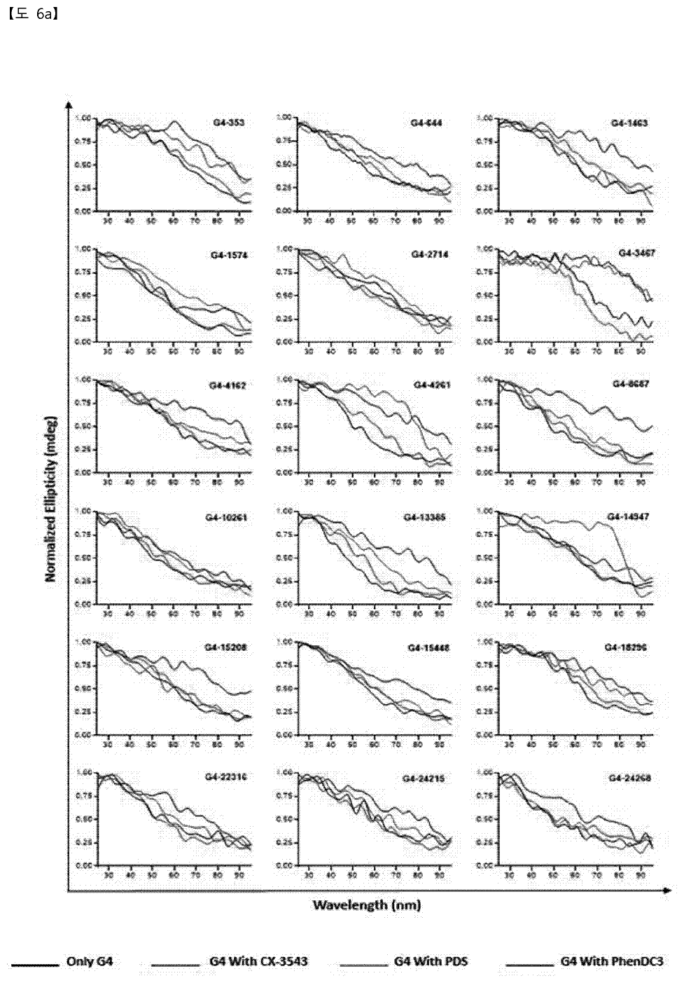

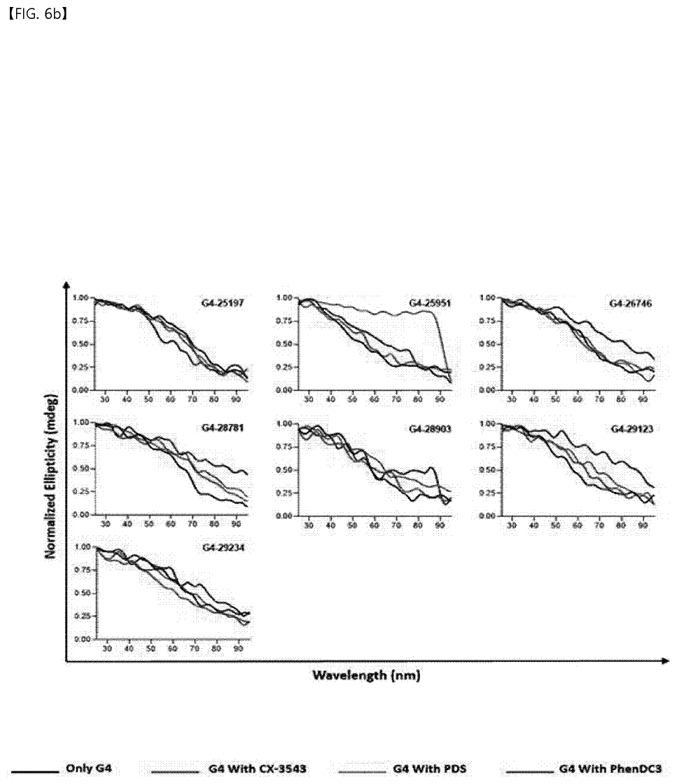

In addition, 15 μM RNA G4 was treated with 30 μM compound and incubated, and a CD thermal melting curve of RNA G4 (15 μM) was obtained in the presence or absence of the G4-binding compound. Furthermore, the effect of G4-binding compounds on the stability of SARS-COV-2 G4 was confirmed by measuring the difference in melting temperature (ΔTm). The results are illustrated in FIGS. 6A and 6B.

As illustrated in FIGS. 6A and 6B, the ΔTm of CX-3543 did not show any noticeable change, whereas the ΔTm of PhenDC3 and PDS was higher than that of CX-3543. This means that PhenDC3 and PDS significantly stabilized most G4s compared to CX-3543. Detailed numerical values are shown in the following Table 4. Further, a comparison of ΔTm between each compound is shown in the following Table 5.

As shown in Table 5, among the 25 G4s predicted for SARS-COV-2, PDS had ΔTm>20° C. in 36% of the G4s, whereas PhenDC3 had ΔTm>20° C. in 72% of the G4s. This means that the proportion of G4s stabilized by the compound is higher for PhenDC3 than for PDS.

TABLE 4

| | | | Tm in | Tm in | Tm in | | | | |
|---|---|---|---|---|---|---|---|---|---|
| G4 | Position | Tm | ° C. with | ° C. with | ° C. with | | | | |
| No. | Gene | (° C.) | CX-3543 | PDS | PhenDC3 | ΔTm_{CX-3543} | ΔTm_{PDS} | ΔTm_{PhenDC3} | Gene Name |
| 1 | 353 | 71.59 | 72.64 | 87.66 | 82.00 | 1.05 | 16.07 | 10.41 | NsP1 |
| 2 | 644 | 48.1 | 53.32 | 70.59 | 71.44 | 5.59 | 22.86 | 23.71 | NsP1 |
| 3 | 1463 | 55.52 | 66.55 | 60.04 | 91.3 | 11.03 | 4.52 | 35.78 | NsP2 |
| 4 | 1574 | 53.38 | 53.43 | NA | 47.74 | 0.05 | NA | −5.64 | NsP2 |
| 5 | 2714 | 71.14 | 57.69 | 86.49 | 91.7 | −13.45 | 15.35 | 20.56 | NsP2 + NsP3 |
| 6 | 3467 | 65.55 | 63.54 | 88.65 | 98.81 | −2.01 | 23.1 | 33.25 | NsP3 |
| 7 | 4162 | 56.89 | 58.94 | 56.03 | NA | 2.05 | −0.86 | NA | NsP3 |
| 8 | 4261 | 50.31 | 63.89 | 79.18 | 85.41 | 13.58 | 28.87 | 35.1 | NsP3 |
| 9 | 8687 | 46.86 | 49.83 | 84.44 | NA | 2.97 | 37.58 | NA | NsP4 |
| 10 | 10261 | 47.89 | 50.24 | 60.66 | 63.49 | 2.35 | 12.77 | 15.6 | NsP5 |
| 11 | 13385 | 45.6 | 52.87 | 63.07 | NA | 7.27 | 17.47 | NA | NsP10 |
| 12 | 14947 | 57.21 | 58.11 | 82.16 | 82.96 | 0.9 | 24.95 | 25.75 | NsP12 |
| 13 | 15208 | 57.17 | 63.87 | 60.16 | NA | 6.7 | 2.99 | NA | NsP12 |
| 14 | 15448 | 52.62 | 58.58 | 56.5 | NA | 5.96 | 3.88 | NA | NsP12 |
| 15 | 18296 | 58.83 | 63.64 | 82.4 | 86.32 | 4.81 | 23.57 | 27.49 | NsP12 |
| 16 | 22316 | 48.28 | 49.56 | 55.62 | NA | 1.28 | 7.34 | NA | Surface glycoprotein |
| 17 | 24215 | 55.42 | 57.16 | 62.93 | NA | 1.74 | 7.51 | NA | Surface glycoprotein |
| 18 | 24268 | 40.47 | 40.42 | 46.11 | NA | −0.05 | 5.64 | NA | Surface glycoprotein |
| 19 | 25197 | 71.19 | 66.57 | 68.63 | 57.51 | −4.62 | −2.56 | −13.68 | Surface glycoprotein |
| 20 | 25951 | 48.74 | 53.87 | 92.26 | NA | 5.13 | 43.52 | NA | ORF3a protein |
| 21 | 26746 | 62.66 | 59.29 | 68.77 | 79.68 | −3.37 | 6.11 | 17.02 | Membrane protein |
| 22 | 28781 | 64.7 | 82.9 | NA | 70.40 | 18.2 | NA | 5.7 | Nucleocapsid protein |
| 23 | 28903 | 57.11 | 63.45 | 60.46 | 60.21 | 6.34 | 3.35 | 3.1 | Nucleocapsid protein |
| 24 | 29123 | 54.4 | 62.59 | 73.83 | NA | 8.19 | 19.43 | NA | Nucleocapsid protein |
| 25 | 29234 | 62.17 | 58.52 | 80.2 | NA | −3.65 | 18.03 | NA | Nucleocapsid protein |

TABLE 5

| | | | |
|---|---|---|---|
| ΔTm range | CX-3543 | PDS | PhenDC3 |

| | | | | |
|---|---|---|---|---|
| 1-5° | C. | 9 | 4 | 1 |
| 5-10° | C. | 7 | 4 | 1 |
| 10-15° | C. | 2 | 1 | 1 |

| | | | |
|---|---|---|---|
| ΔTm 15-20° C. | 1 | 5 | 2 |
| ΔTm >20° C. | 0 | 9 | 18 |
| Destabilized | 6 | 2 | 2 |

### Example 5. Confirmation of Cytotoxicity

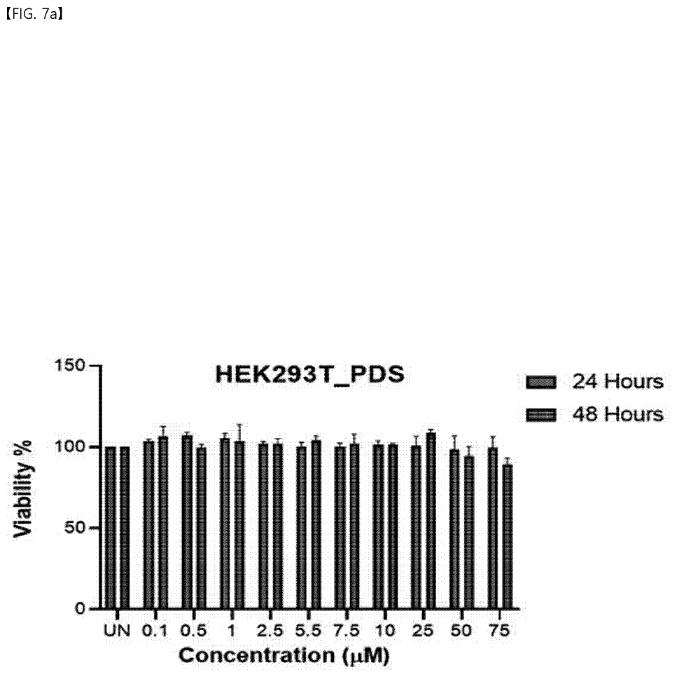

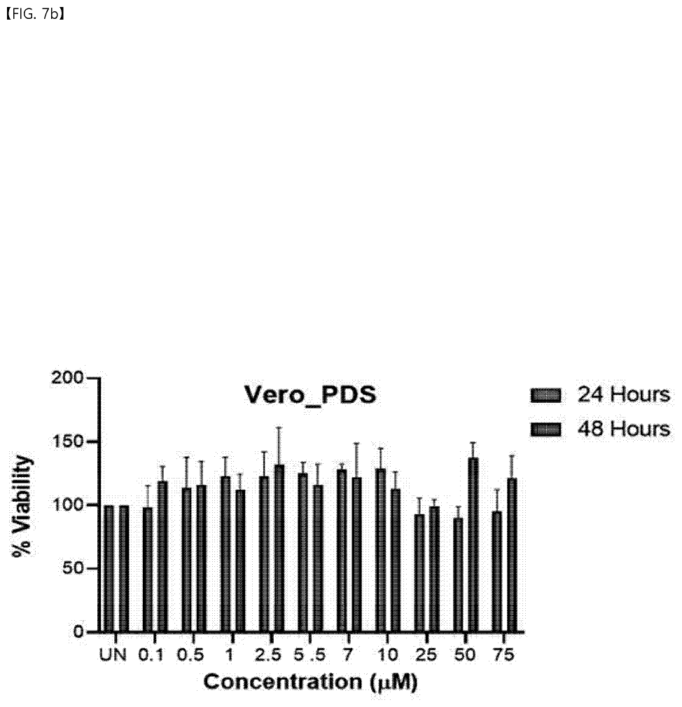

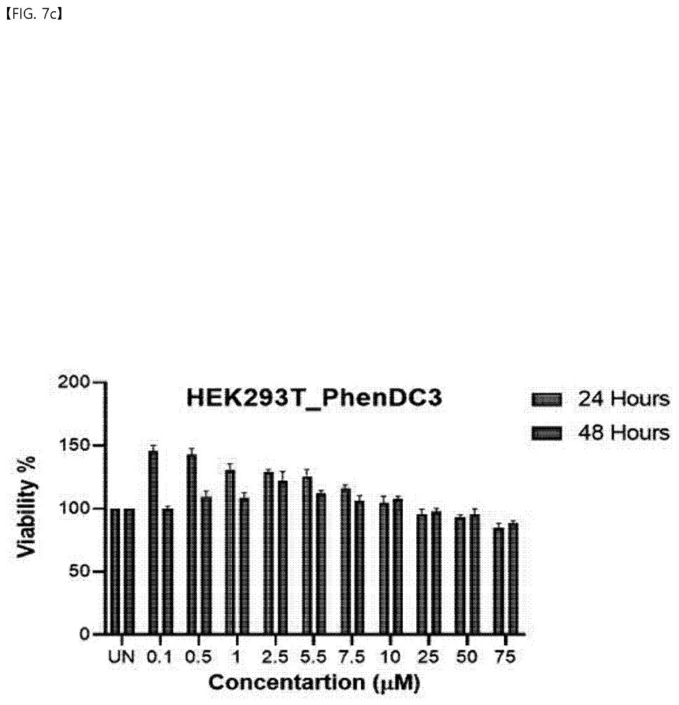

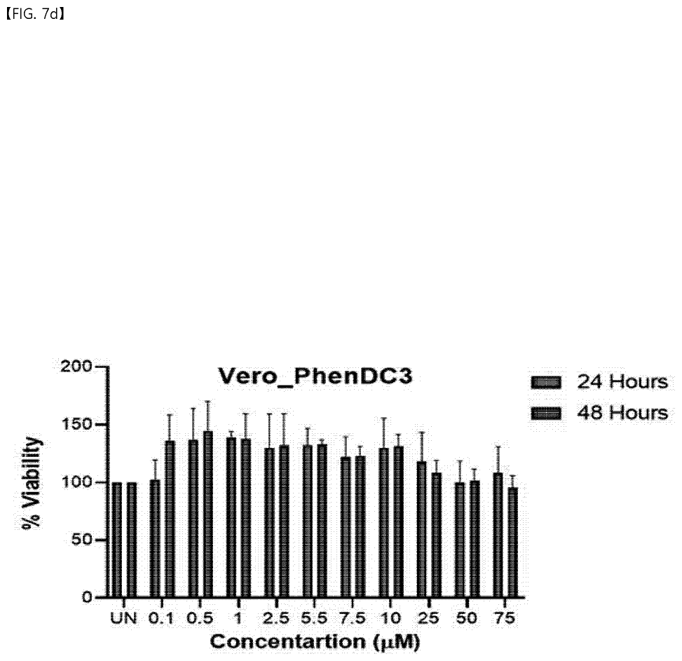

The toxicity of PDS and PhenDC3 was confirmed in HEK293T cells and Vero cells. The results are illustrated in FIGS. 7A to 7D.

As illustrated in FIGS. 7A and 7C, PDS was safe to use up to 75 μM in HEK293T cells, but PhenDC3 at 75 M had a 25% reduction in viability after 48 hours. In addition, as illustrated in FIGS. 7B and 7D, no cytotoxicity was observed for PDS and PhenDC3 up to 75 μM after 48 hours in Vero cells.

### Example 6. PCR Amplification of WT-G4 and Mut-G4 Comprising T7 Promoter

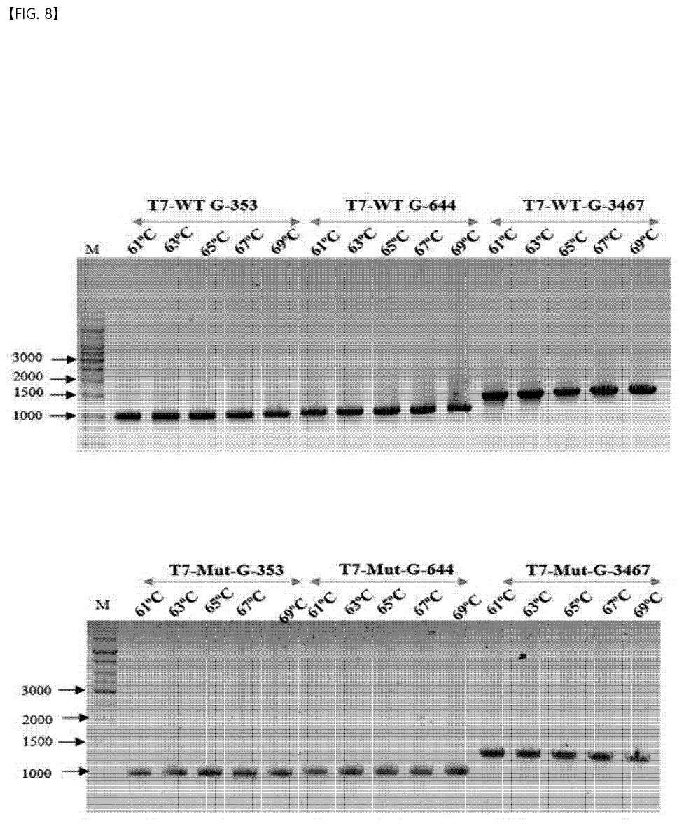

For in-vitro transcription/translation (IVT) analysis, N₁-WT-G4-353, 644, and 3467 and N₁-Mut-G4-353, 644, and 3467 were PCR amplified with the T7 promoter site, and named T7-WT-G4-353, 644, and 3467 and T7-Mut-G4-353, 644, and 3467, respectively. The results are illustrated in FIG. 8.

As illustrated in FIG. 8, the distinct single bands of amplified products eluted from the gel at 65° C. were observed.

### Example 7. In-Vitro Transcription/Translation (IVT) Analysis

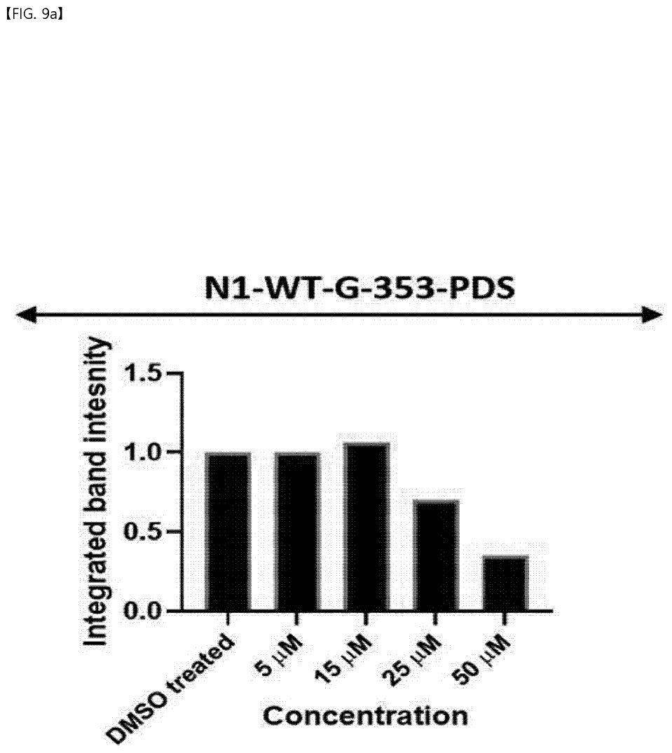

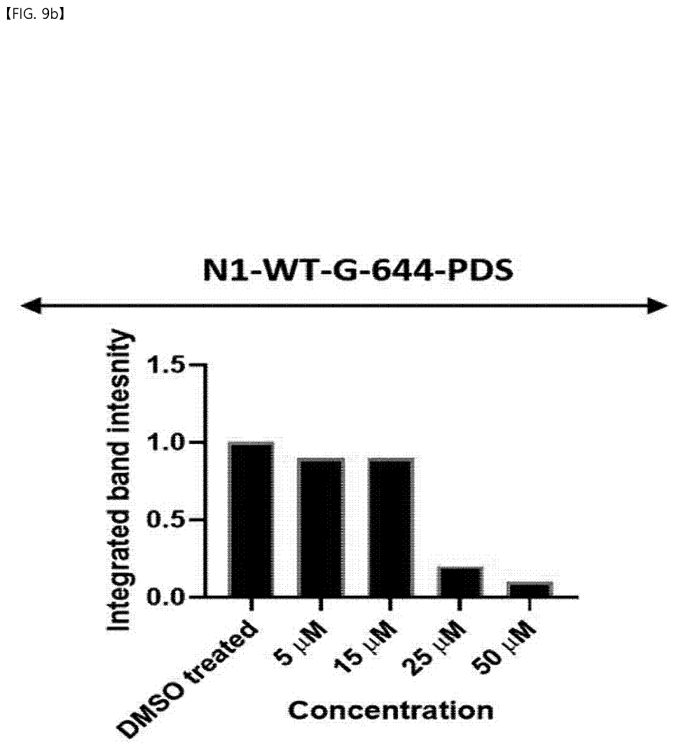

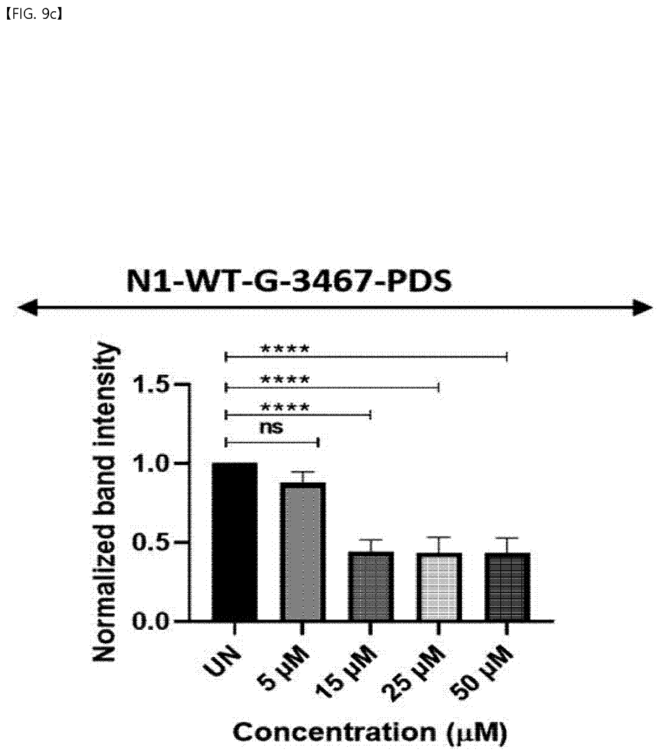

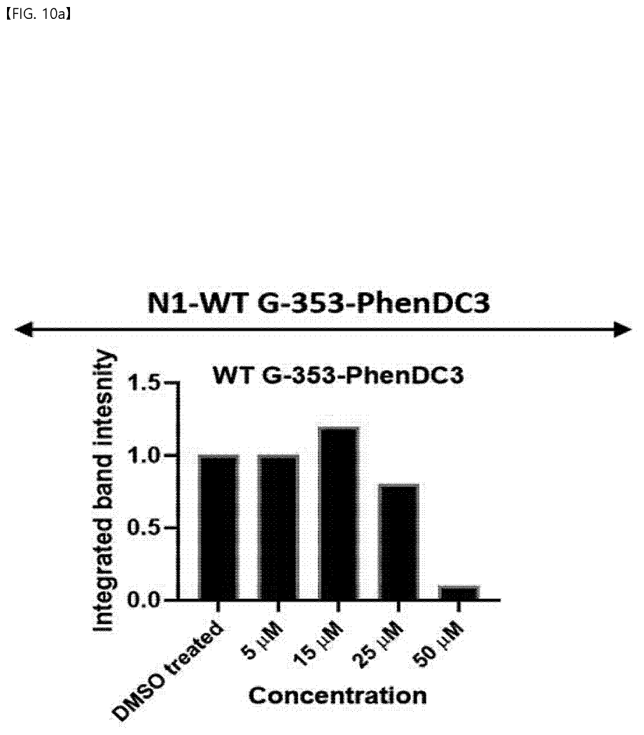

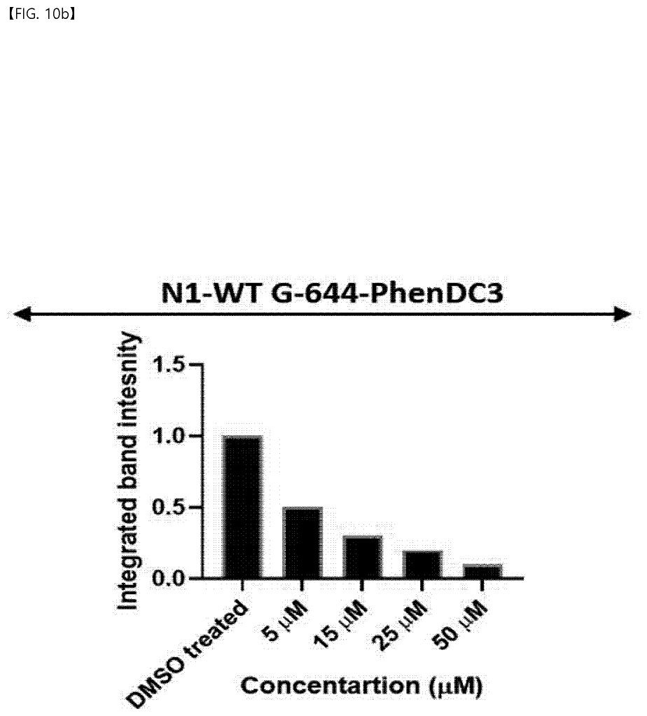

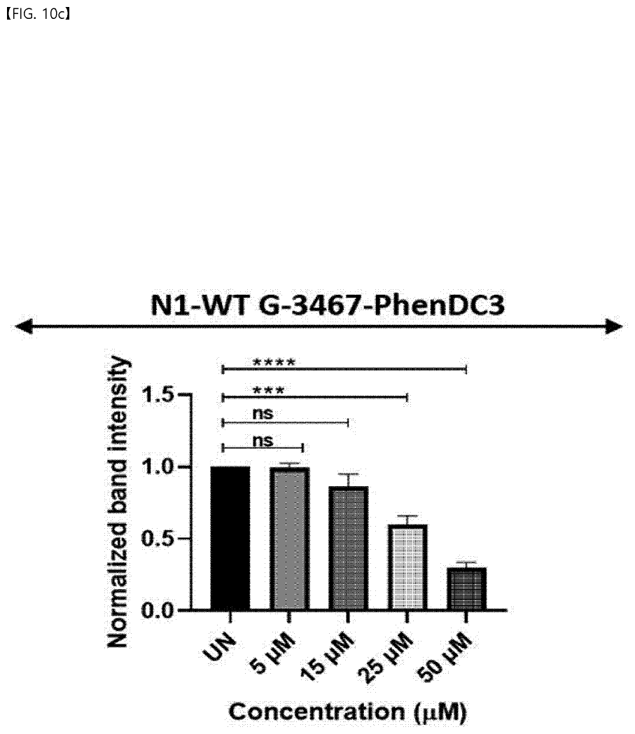

By IVT analysis, the effect of various concentrations of G4-binding compounds on translation was confirmed. When translation is suppressed, it means that the corresponding compound is effective for stabilizing the G4 structure. In order to visualize this, an enhanced yellow fluorescent protein (EYFP) reporter system was constructed, and 48 hours after compound treatment, cells were harvested to analyze EYFP expression by western blot. The results are illustrated in FIGS. 9A to 9C, 10A to 10C, 11A to 11D, and 12A to 12D.

As illustrated in FIGS. 9A, 10A, 11A and 12A, EYFP expression was reduced by 65% and 90% when N1-WT-G4-353 was treated with PDS and PhenDC3 at 50 μM, respectively. Concentrations less than 50 μM were not effective for suppressing EYFP reporter expression.

As illustrated in FIGS. 9B, 10B, 11B and 12B, EYFP expression was reduced by 80 to 90% when N1-WT-G4-644 was treated with PDS at 25 and 50 μM, but even when N1-WT-G4-644 was treated with PhenDC3 at 5 μM, EYFP expression was reduced by 50%.

As illustrated in FIGS. 9C, 10C, 11C and 12C, when N1-WT-G4-3467 was treated with PDS at 15 to 50 μM, EYFP expression was suppressed by 60% and no change was observed according to an increase in drug concentration. When N1-WT-G4-3467 was treated with PhenDC3 at 50 μM, a concentration-dependent decrease was exhibited along with a 70% reduction in EYFP expression.

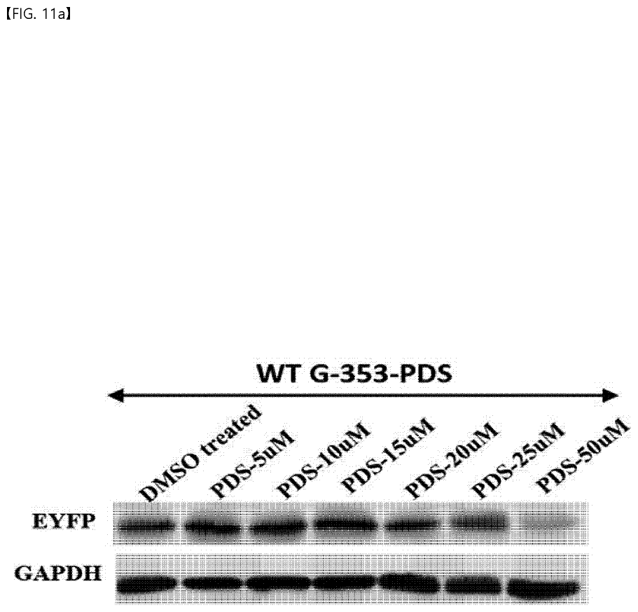

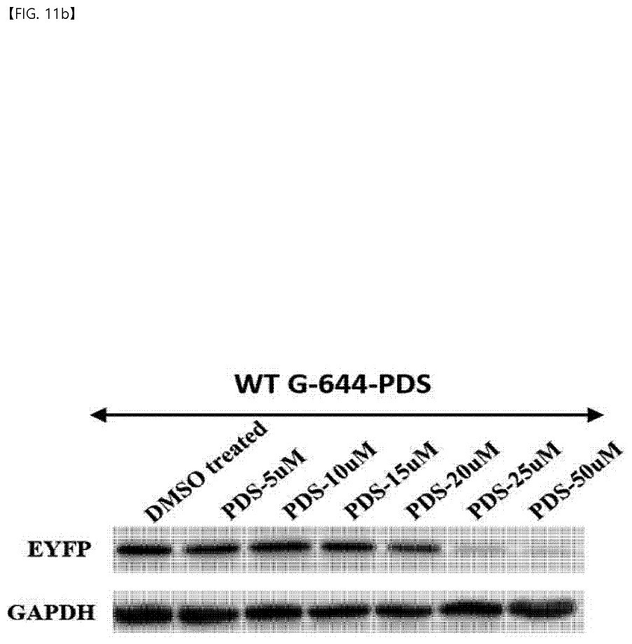

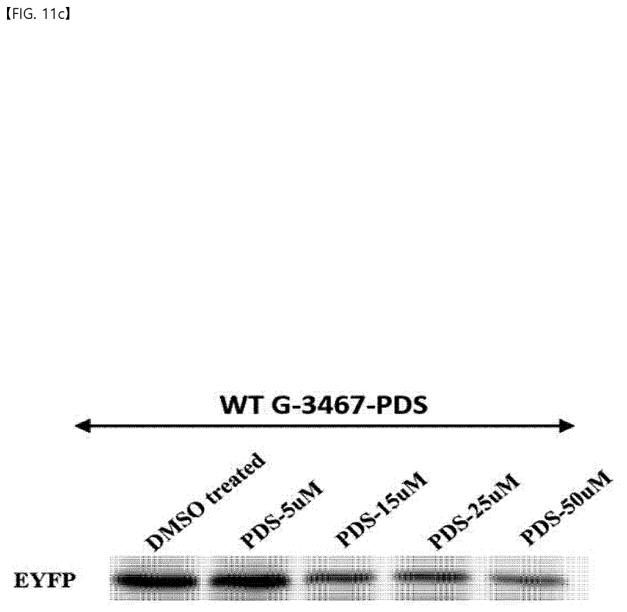

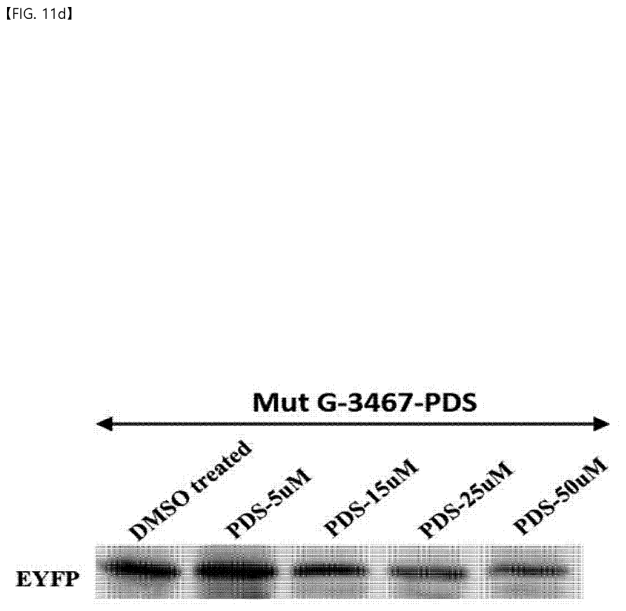

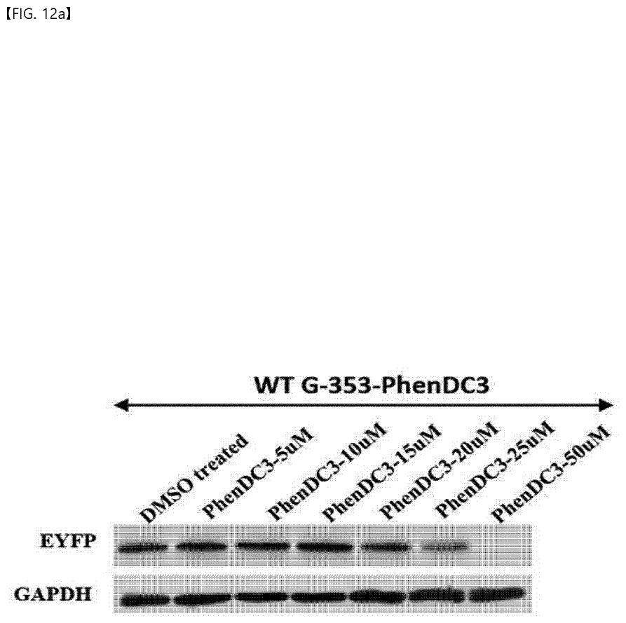

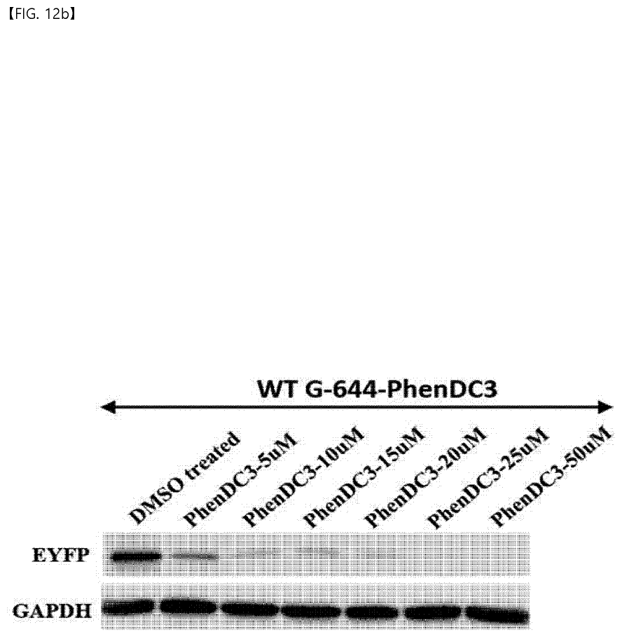

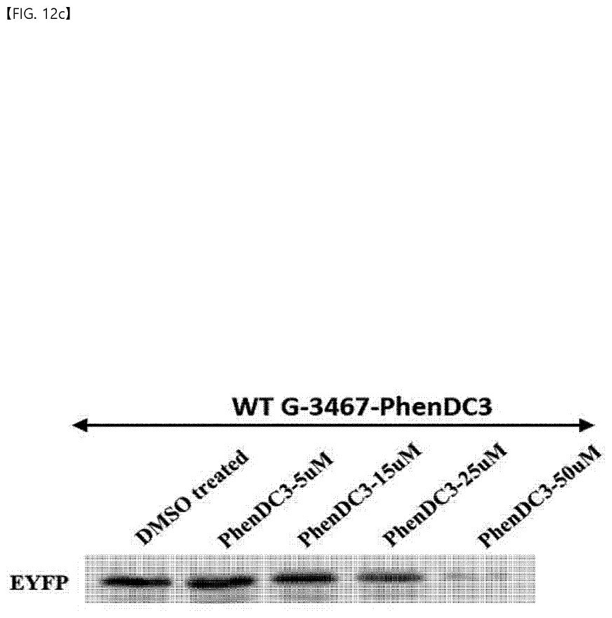

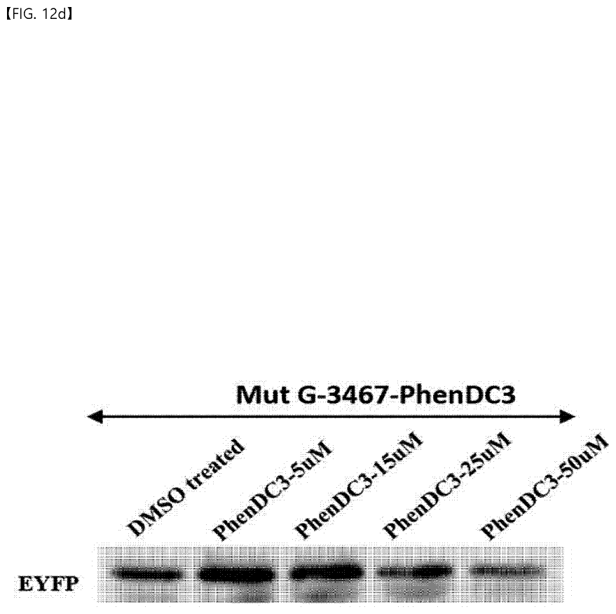

As illustrated in FIGS. 11D and 12D, when N1-Mut-G4-3467 was treated with PDS and PhenDC3, EYFP expression was not suppressed in a concentration-dependent manner.

The results mean that PDS and PhenDC3 suppress the translation of Nsp1 and Nsp3 mRNAs by stabilizing the G4 structures of coronaviruses. In addition, the results mean that PhenDC3 is more effective for stabilizing SARS-COV-2 G4 than PDS.

### Example 8. Confirmation of Antiviral Efficacy of G4-Binding Compounds Against SARS-COV-2-N Protein Expression Relationship

The N protein is one of the structural proteins of coronaviruses and plays an important role in the viral packaging, viral nucleation and viral RNA synthesis of coronaviruses. To confirm the antiviral activity of the compounds, the extent of N protein expression in infected cells was analyzed.

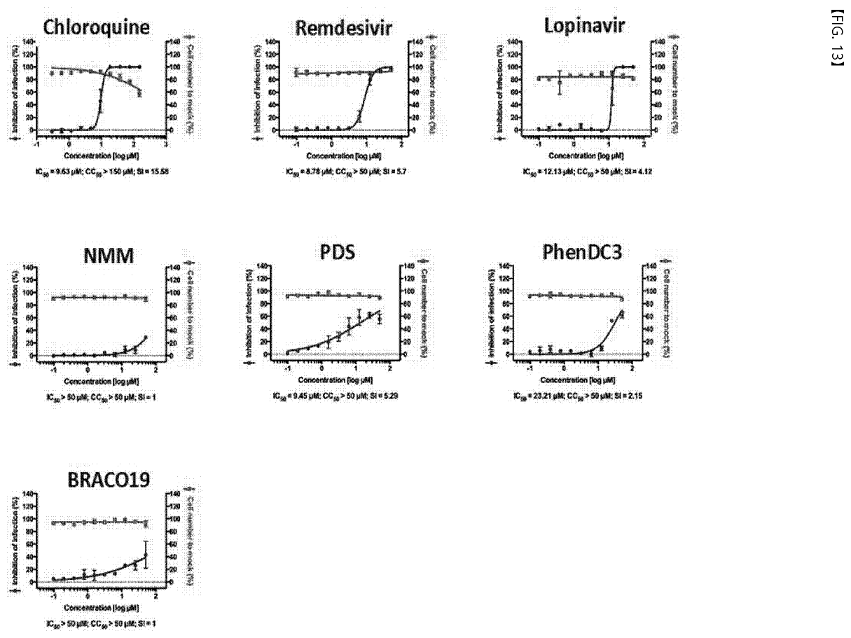

As illustrated in FIG. 13, among the G4-binding compounds, PDS and PhenDC3 were the most effective for suppressing SARS-COV-2 infection with an IC₅₀ of 9.45μ M and 23.21μ, respectively. Furthermore, NMM and BRACO19 suppressed SARS-COV-2 infection at high concentrations. In particular, the IC₅₀ of PDS was similar to positive controls such as chloroquine (9.63 μM), remdesivir (8.78 μM), and lopinavir (12.13 μM), implying that PDS exhibits potent antiviral activity against SARS-COV-2.

### Example 9. Confirmation of Antiviral Efficacy of G4-Binding Compounds Against SARS-COV-2-Virion Production Relationship

Virions are complete viral particles consisting of a capsid and nucleic acid, and virion production is associated with the smoothness of viral replication. To confirm the antiviral efficacy of the compounds, virion production was analyzed by quantifying the RNA genome of the SARS-COV-2 virus present in the supernatant.

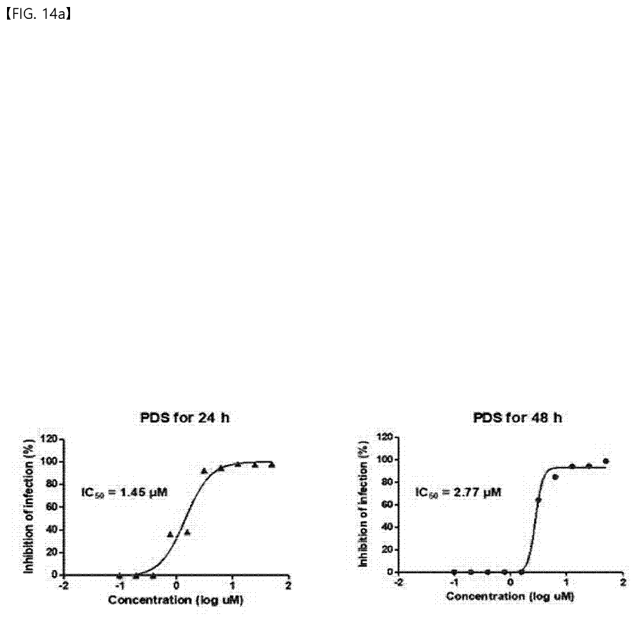

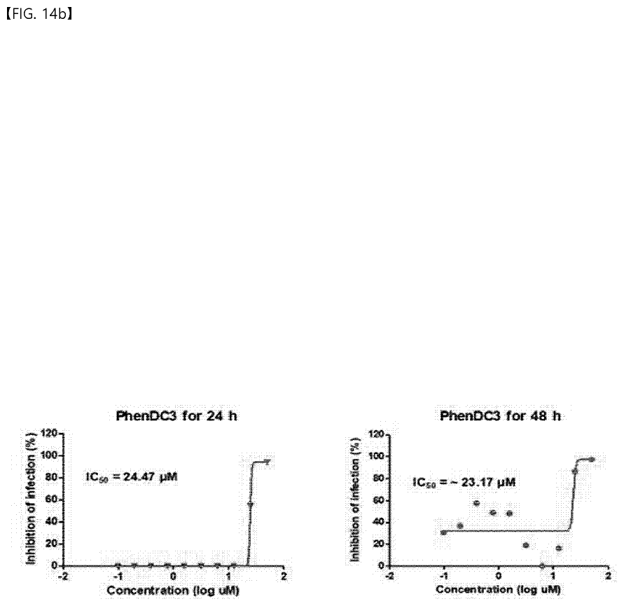

As illustrated in FIG. 14, PDS exhibited potent antiviral activity against virion production with IC₅₀ values of 1.45 μM and 2.77 μM at 24 hours and 48 hours after infection, respectively. PhenDC3 also suppressed SARS-COV-2 virion production, and IC₅₀ values were estimated to be 24.47 μM and 23.17 μM at 24 hours and 48 hours after infection, respectively. As in the results in Example 8, this suggests that PDS and PhenDC3 are potent in suppressing SARS-COV-2 replication in Vero cells.

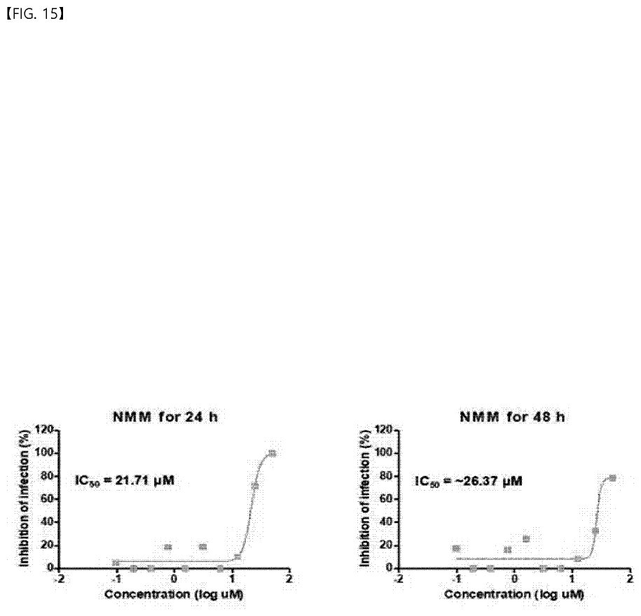

As illustrated in FIG. 15, since the IC₅₀ of NMM was similar to that of PhenDC3, NMM was effective for suppressing the virion production of SARS-COV-2 in Vero cells.

In summary, it means that the G4-binding compounds, PDS, PhenDC3 and NMM, have an excellent effect of suppressing SARS-COV-2 replication by stabilizing the G4 structure in SARS-COV-2 transcripts.

### Example 10. Confirmation of Antiviral Efficacy of G4-Binding Compounds Against HCoV-OC43 Virus-Virion Production Relationship

Virions are complete viral particles consisting of a capsid and nucleic acid, and virion production is associated with the smoothness of viral replication. Thus, to confirm the antiviral efficacy of the G4-binding compounds, virion production was analyzed by quantifying infectious virus present in the supernatant by plaque assay.

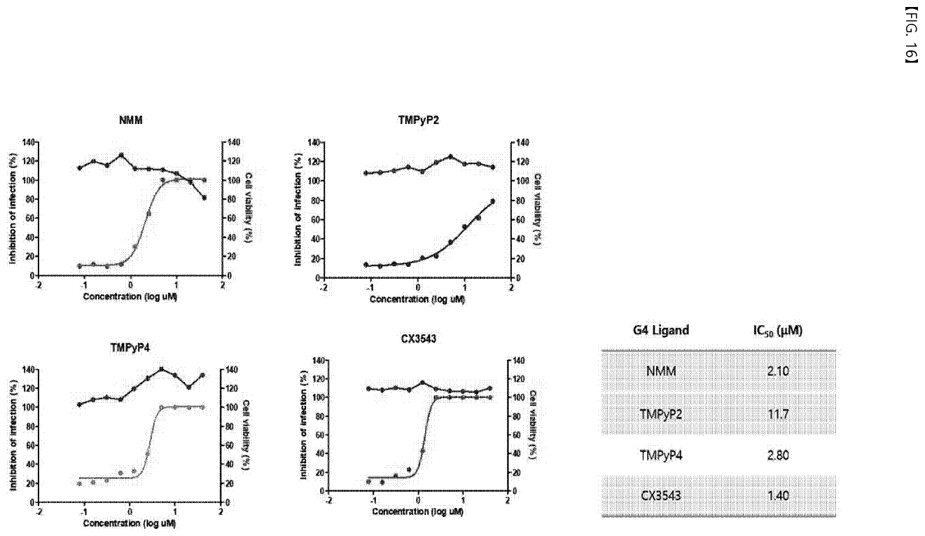

As illustrated in FIG. 16, NMM exhibited potent antiviral activity against virion production with IC₅₀ values of 2.10 μM after infection. The G4-binding compounds, TMPyP2, TMPyP4 and CX3543, also suppressed the virion production of the HCoV-OC43 virus, and IC₅₀ values were estimated to be 11.7 μM, 2.80 μM, and 1.40 μM after infection, respectively. This suggests that CX3543, NMM, TMPyP4 and TMPyP2 are potent in suppressing HCoV-OC43 viral replication in Vero cells.

This means that the G4-binding compounds, NMM, TMPyP2, TMPyP4, and CX3543, have an excellent effect of suppressing HCoV-OC43 virus replication by stabilizing the G4 structure of the HCoV-OC43 virus.

Therefore, it is expected that the G4 structure-binding compounds of the present invention can be used to treat the infectious diseases of coronaviruses comprising SARS-COV-2 and HCoV-OC43 viruses.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

Since the compound according to the present invention can bind to G4 structures of coronaviruses to stabilize the G4 structure, reduce the protein expression of coronavirus, and effectively inhibit cell infection by viruses, the compound can be used in the prevention or treatment of coronavirus infectious diseases, and thus has industrial applicability.

1. A method for treating coronavirus infectious diseases, the method comprising administering a pharmaceutical composition comprising, as an active ingredient, a compound that binds to G4 structures of coronaviruses or a pharmaceutically acceptable salt thereof to a subject in need thereof.

2. The method of claim 1, wherein the compound is one or more selected from the group consisting of compounds represented by the following Chemical Formulae 1 to 7.

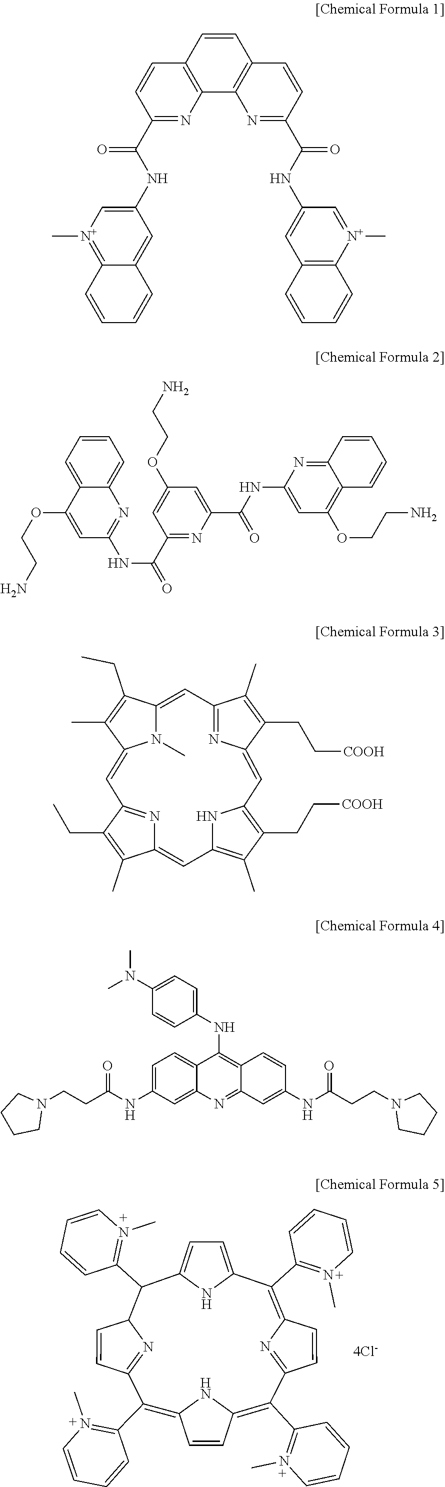

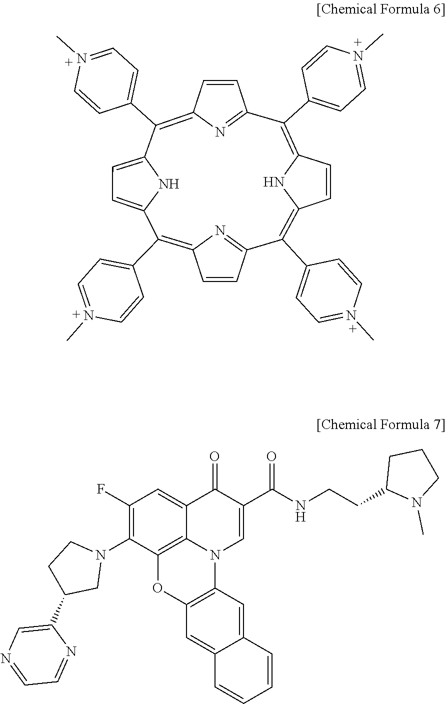

3. The method composition of claim 1, wherein the coronavirus is one or more selected from the group consisting of human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, Middle East respiratory syndrome coronavirus (MERS-COV), and severe acute respiratory syndrome coronavirus 2 (SARS-COV-2).

4. The method of claim 1, wherein the compound stabilizes the G4 structures of coronaviruses.

5. The method composition of claim 1, wherein the compound suppresses the expression of one or more proteins selected from the group consisting of coronavirus protein non-structural protein 1 (Nsp1), non-structural protein 3 (Nsp3), and a nucleocapsid protein.

6. The method of claim 1, wherein the compound suppresses coronavirus virion production.

7. An antiviral composition against coronaviruses, comprising a compound that binds to G4 structures of coronaviruses as an active ingredient.

8. The food composition of claim 7, wherein the compound is one or more selected from the group consisting of compounds represented by the following Chemical Formulae 1 to 7.

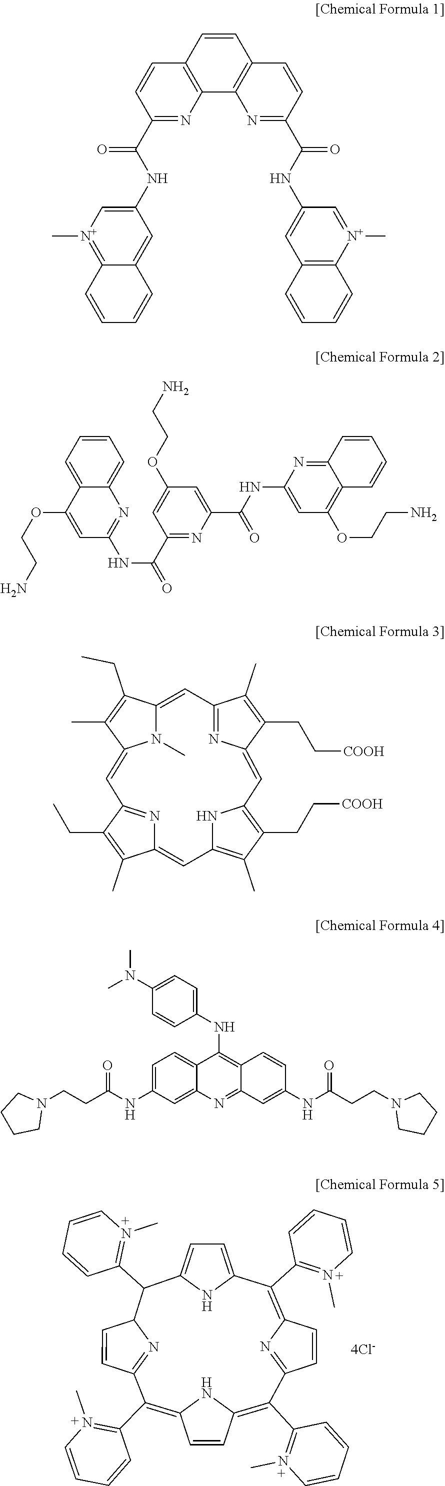

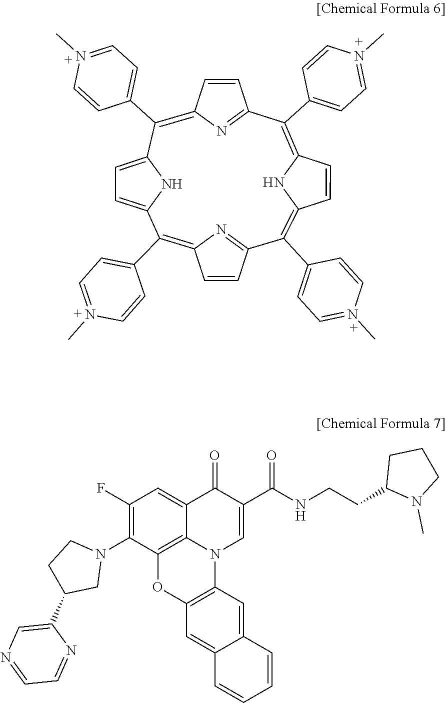

9-13. (canceled)

14. The composition of claim 7, wherein the coronavirus is one or more selected from the group consisting of human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, Middle East respiratory syndrome coronavirus (MERS-COV), and severe acute respiratory syndrome coronavirus 2 (SARS-COV-2).

15-20. (canceled)

21. A method for suppressing coronaviruses, the method comprising applying a pharmaceutical composition comprising, as an active ingredient, a compound that binds to G4 structures of coronaviruses or a pharmaceutically acceptable salt thereof to a subject.

22. The method of claim 21, wherein the compound is one or more selected from the group consisting of compounds represented by the following Chemical Formulae 1 to 7.

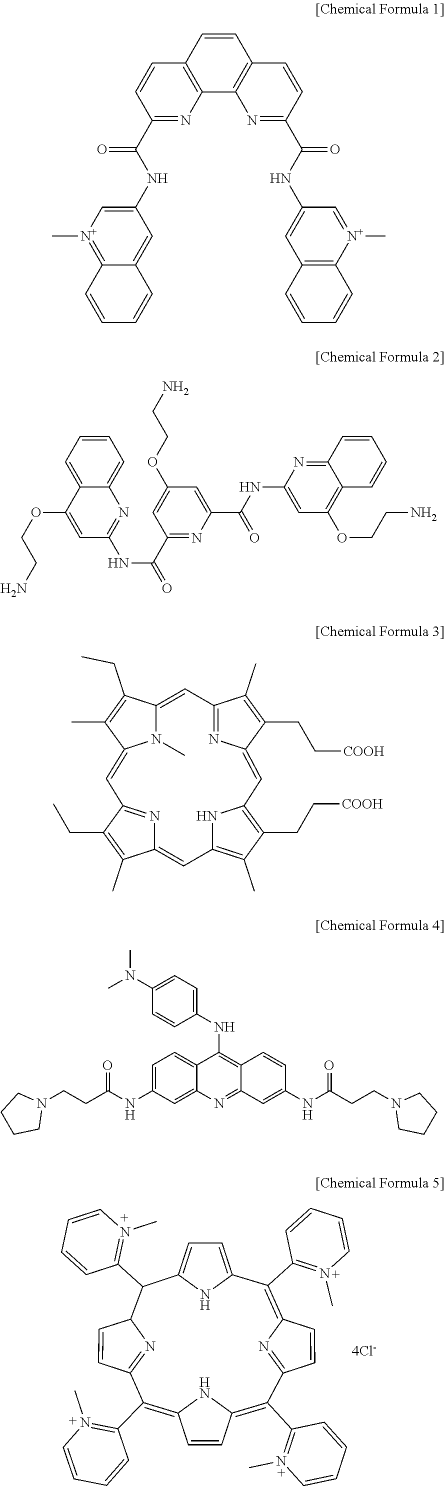

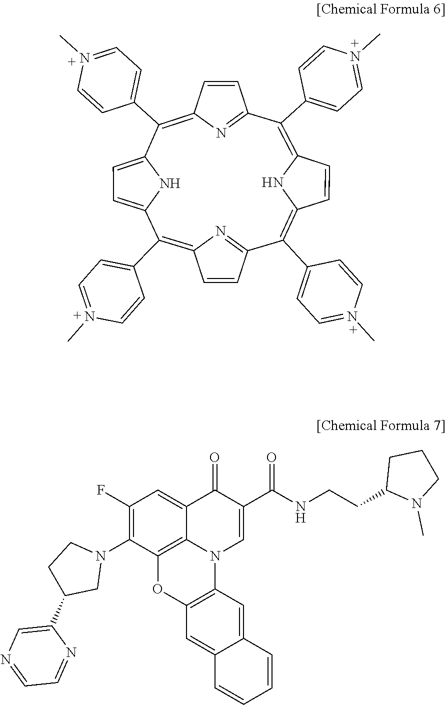

23. The method of claim 21, wherein the coronavirus is one or more selected from the group consisting of human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKU1, Middle East respiratory syndrome coronavirus (MERS-COV), and severe acute respiratory syndrome coronavirus 2 (SARS-COV-2).

24. The method of claim 21, wherein the compound stabilizes the G4 structures of coronaviruses.

25. The method composition of claim 21, wherein the compound suppresses the expression of one or more proteins selected from the group consisting of coronavirus protein non-structural protein 1 (Nsp1), non-structural protein 3 (Nsp3), and a nucleocapsid protein.

26. The method of claim 21, wherein the compound suppresses coronavirus virion production.
